(19) United States
(12) Patent Application Publication
(10) Pub. No.: US 2024/0141043 A1
(43) Pub. Date: May 2, 2024

(54) BISPECIFIC ANTIBODY THAT BINDS TO CD3 AND A FLUOROPHORE

(71) Applicants: ETH ZURICH, Zurich (CH); UNIVERSITÄT ZÜRICH, Zürich (CH)

(72) Inventors: Dario NERI, Lachen (CH); Renier MYBURGH, Aarau (CH); Markus MANZ, Zürich (CH); Laura VOLTA, Zürich (CH)

(21) Appl. No.: 18/273,602

(22) PCT Filed: Jan. 24, 2022
(86) PCT No.: PCT/EP2022/051477

§ 371 Date: Jul. 21, 2023

(30) Foreign Application Priority Data
Jan. 22, 2021 (EP) 21152969.8

(51) Int. Cl. C07K 16/28; A61K 47/68; A61K 49/00; A61P 35/00; C07K 16/44

(52) U.S. Cl. CPC C07K 16/2809; A61K 47/6897; A61K 49/0058; A61P 35/00; C07K 16/44; A61K 2039/505; C07K 2317/31

(57) ABSTRACT

The present invention relates to a bispecific antibody comprising at least a first binding domain and a second binding domain, wherein said first binding domain binds to an organic fluorophore and wherein said second binding domain binds to CD3, and to a combination thereof with a labelled binding agent that binds specifically to a target antigen, wherein the labelled binding agent is labelled with an organic fluorophore.

### FIELD OF THE INVENTION

The present invention relates to a bispecific antibodies.

### BACKGROUND

Bispecific, T-cell engaging and activating antibodies and chimeric antigen receptor (CAR) T-cells (CAR T) cells are promising antibody-based approaches to treat various types of cancers (or other diseases as autoimmunity, inflammation or infection). Both therapies rely on the (re)direction of the killing activity of T cells towards target cells.

Bispecific antibodies are biopharmaceuticals, able to simultaneously bind two different antigens with the intention to link an effector cell (e.g. T-cell) to a target-antigen expressing cell, leading to effector-cell mediated killing of the target-antigen carrying cell. Specifically, bispecific antibodies can bind CD3 on T-cells and a tumor-associated antigen, bridging together tumor cells and effector T-cells. The cross-linking leads to T-cell activation and elicits a potent and selective (re)targeting of their cytolytic activity to the target-carrying cell in an MHC-independent manner. Clinical-stage bispecific antibodies range from small proteins, consisting of two linked antigen-binding fragments, to large immunoglobulin G (IgG)-like molecules with additional domains attached.

CAR-T cells are genetically modified T-cells that overexpress an artificial CAR which usually is comprised of a single-chain variable fragment (scFv) binding domain linked to the T-cell via a flexible hinge and a transmembrane domain. Additional CAR components, the intracellular co-stimulatory and stimulatory domains, mediate the activation signal. The scFv on the surface re-directs CAR-T cell cytotoxic activity in an MHC-independent manner against the target cells where the cognate antigen is expressed.

One bispecific T-cell engager (BiTE™), consisting of two scFvs against CD3 and CD19 has gained regulatory approval (Blinatumomab; Blycinto®) as well as two CAR-T cells products (Kymriah® and Yescarta®) as salvage therapies for certain subtypes of B-cell leukemia or lymphoma.

However, despite high rates of initial response, relapse is observed in a notable proportion of patients. Bispecific antibodies and CAR-T cells, capable of targeting a single tumor cell antigen, are unable to address antigen escape or overcome tumor heterogeneity. Outcome improvement by use of combinatorial strategies would require the development of a new GMP product for each new antigen one may want to target.

Compared to the development of a recombinant protein, GMP production of CAR-T cells accounts for the costs of a personalized medicine, where each batch is manufactured from patients' cells, thus limiting its clinical applicability. Moreover, while the activity of the antibodies can be controlled by injection schedule, this on-off safety switch is currently not possible when using CAR-T cells. Their activation lacks downstream control mechanisms after injection which may result in severe side effects derived from systemic toxicities or on-target, off-tumor effects due to the strong level of CAR T-cell activation.

However, so far, only a small selection of bispecifc T cell engaging binding agents is available on the market. This severely restricts the clinical potential of T cell engaging therapy to diseases that exhibit targets for which one of the few bispecifc T cell engaging binding agents is already available, and, in case the respective disease develops a resistance against said therapy, leaves the practitioner with no suitable 2^{nd} line therapy.

### SUMMARY OF THE INVENTION

The present invention provides a bispecific antibody comprising at least a first binding domain and a second binding domain, wherein said first binding domain binds to an organic fluorophore and wherein said second binding domain binds to CD3, and to a combination thereof with a labelled binding agent that binds specifically to a target antigen, wherein the labelled binding agent is labelled with an organic fluorophore.

Such new constructs are called “Universal T cell engaging and activating bispecific antibodies”, abbreviated UniTEA herein.

### BRIEF DESCRIPTION OF THE FIGURES

FIG. 1. Schematic representation and characterization of UniTEAs.

(A) The Universal T-cell engaging and activating bispecific antibody (UniTEA) has dual specificity for human CD3 (e.g. CD3ε) expressed on T-cells and for fluorescein. T-cell cytotoxic activity is only elicited in presence of a fluorophore-tagged, target-bound adaptor. The fluorophore is shown as a star ( [symbol] ). Different types of binders are shown that can be used as adaptors, namely an antibody (left part, symbolized by the IgG-like shape, yet also encompassing other fragments or derivatives thereof, like e.g. a scFv or diabody, as well as a small molecule (middle part) or a peptide (right part)),

(B) Size exclusion chromatography profiles and SDS PAGE gels showing the purity and molecular weight of three different UniTEA formats, called 4m5.3/BlinCD3, E2/BlinCD3 and FluA/BlinCD3.

FIG. 2. T cell-mediated depletion of CD19, CAIX and CD117-expressing cell lines in vitro.

(A) NALM-6 (CD19⁺)B-ALL cell line was used to compare the T cell mediated killing elicited by the 4m5.3/BlinCD3 and FluA/BlinCD3 UniTEA formats to Blinatumomab™ (anti-CD19/BlinCD3 T-cell Engaging and Activating bispecific antibody, TEA). Target cells and T-cells were co-incubated for 24 hrs and 48 hrs at 10:1 E:T ratio.

(B) Background killing of only UniTEA or commercial anti human CD19 IgG-FITC adaptor molecule at the indicated concentrations.

(C) Equimolar concentration of 4m5.3/BlinCD3 and FluA/BlinCD3 UniTEAs elicited similar T-cell mediated target cell lysis as Blinatumomab™ when 10 nM of adaptor were added.

(D) Renal cell carcinoma cell line SK-RC-52 (CAIX⁺) was used to assess the T cell mediated killing elicited by 4m5.3/BlinCD3, FluA/BlinCD3 and E2/BlinCD3 UniTEAs in presence of 10 nM site-specifically fluoresceinated anti-CAIX IgG.

(E) All three UniTEA formats induced T-cell mediated tumor cell lysis in a concentration-dependent fashion after 24 hrs at 10:1 E:T ratio. Background killing after co-culturing T- and tumor cells alone or by adding only 10 nM adaptor molecule is also reported.

(F) A comparison between CAR T cell, TEA and UNiTEA was performed by targeting CD117 on MOLM14 (CD117 k) AML cells. The 4m5.3/BlinCD3 UNiTEA (BiTE format) was used in combination with a site-specifically fluoresceinated anti-CD117 diabody (Db). Target cells and effector cells were co-incubated for 24 hrs and 48 hrs at 1:1 E:T ratio.

(G) Addition of 4m5.3/BlinCD3 UniTEA in presence of 10 nM fluorescinated anti-CD117 Db resulted in similar T-cell mediated target cell lysis at 24 and 48 hours as direct anti-CD117 CAR-T cells and anti-CD117/BlinCD TEA.

FIG. 3. In vivo persistence of FITC-antibodies.

(A) Commercially available anti murine CD19 (mCD19) IgG-FITC was injected i.v. in two C57BL/6J mice (50 ug/mouse). Peripheral blood was analyzed by FACS at different time points over 8 days.

(B) Mean fluorescence intensity (MFI) of mCD19⁺ cells stained at each time point with and without additional mCD19 IgG-FITC, represented by filled and hollow circles, respectively.

(C) Live mCD19⁺/mCD20⁺ B cells, expressed as percentage of mCD19⁺/mCD20⁺ cells at each time point compared to the baseline number after 4 hrs, stained with and without additional mCD19 IgG-FITC, represented by filled and hollow circles, respectively.

(D) Representative FACS plots showing the peripheral blood CD19⁺CD20⁺ murine B cell population at indicated time points with and without additional CD19-FITC labelling.

FIG. 4: Structures of FITC and Fluorescein-5-Maleimide (the latter is used for site specific conjugation of FITC to an antibody that is labelled with a terminal Cystein residue)

FIG. 5: Overview of different bispecific antibody formats. This figure shows the different formats of bispecific antibodies that can be used in the present invention. It has been taken from Spiess et al. 2015, the content of which is incorporated herein by reference, in particular to account for a potential loss of color information.

FIG. 6: T cell-mediated depletion of PSMA+LNCaP human prostate adenocarcinoma cells by fluoresceinated PSMA targeting ligand and E2/OKT3 UniTEAs in vitro.

(A) The UniTEA has dual specificity for human CD3ε (OKT3 binder) expressed on T-cells and for fluorescein (E2 binder). T-cell cytotoxic activity against PSMA+ cells is only elicited in presence of a fluorescein-tagged, PSMA-specific adaptor, i.e. the small molecule DUPA-FITC (D-F).

(B) Representative FACS plots showing PSMA+ target cells co-cultured with T cells, 1 nM UniTEA and indicated concentrations of DUPA-FITC (D-F) for 24 hr.

(C) Quantification of the percentage specific lysis of PSMA+CD3− cells achieved 24 hours after co-culture with T cells (E:T 10:1), 1 nM UniTEA and indicated concentrations of DUPA-FITC

(D-F). Specific lysis was calculated as follows: (1-number of alive target cells/number of alive target cells without antibody)*100.

FIG. 7. T cell-mediated depletion of double positive CD20+/CD38+ human B cell lymphoma cells with fluoresceinated, clinically approved antibodies and E2/OKT3 UniTEAs in vitro.

(A) The UniTEA has dual specificity for human CD3ε (OKT3 binder) expressed on T-cells and for fluorescein (E2 binder). T-cell cytotoxic activity against CD20+/CD38+ cells is only elicited in presence of a fluorescein-tagged, anti-CD20 or anti-CD38 clinically approved antibodies.

(B) Representative FACS plots showing the cell surface expression of human CD3, CD19, CD20 and CD38 on DOHH-2 NHL cell line.

(C) Flow cytometry histograms showing DOHH-2 cells labelled with indicated concentrations of clinically available anti-CD20 (Rituximab) and anti-CD38 (Daratumomab) human IgG1 labelled with FITC (fluorescein:protein ratio=2).

(D) Representative FACS plots showing the proportion of CD19+CD3− cells left over 24 hours after co-culture with T cells (E:T 1:1) and indicated antibodies. We use CD19 to identify the target cells since the DOHH2 cells are positive for CD19, CD20 and CD38 and this avoids epitope blockade of the targeting antibodies and detection antibodies (FIG. 5B).

(E) Quantification of the percentage specific lysis of CD3−CD19+CD2O+CD38+DOHH-2 cells achieved 24 hours after co-culture with T cells and indicated antibodies.

FIG. 8. T cell-mediated depletion of human AML cell lines is enhanced by combinatorial targeting of tumor-associated antigens using a combination of fluoresceinated tumor-specific binders and UniTEAs in vitro.

(A) Cartoon shows target cell populations that express antigen A or antigen B, or both antigen A and B. Fluorescinated adaptors against antigen A or B will bind to respective cells via the expressed antigens. This leads to higher labeling and therefore more fluorescine expression of cells that express both, antigen A and B. Addition of UniTEA will thus lead to enhanced T-cell activation and killing of cells that express both antigen A and B, while killing of cells that only express either antigen A or B will be less efficient. This system thus allows enhanced killing of select multi-targeting antigen positive populations by combinatorial use of linkers, while at the same time single-target antigen expressing cells are less efficiently killed.

(B) Representative FACS plots showing the cell surface expression of CD117 and CD371 on a mixture of MOLM13 human AML cell lines transduced to express either CD117 (lower right corner), CD371 (upper left corner) or both CD117 and CD371 (upper right corner). In addition, CD117 and CD371 negative T-cells in the cellular mixture are shown (lower left corner).

(C) Quantification of percentage specific lysis of CD33+CD3−MOLM13 cells after being co-cultured for 24 hours with T cells at the indicated concentration of E2/OKT3 UniTEAs and fluoresceinated tumor antigen specific adaptors used in combination and as single agents (E:T 1:10).

(D) Representative FACS plots showing the proportion of CD33+CD3− cells left over 24 hours after co-culture with T cells, indicated fluoresceinated tumor antigen specific adaptors and UniTEAs.

FIG. 9. T cell-mediated depletion of human AML cell lines, engrafted in immunodeficient NSG mice treated with fluoresceinated tumor antigen specific adaptors and UniTEAs.

(A) Experimental scheme of preventive in vivo AML treatment, using AML cell-lines, human primary T-cells as well as UniTEA and fluorescinated diabodies.

(B) Quantification of live bioluminescence imaging of immunodeficient mice after 7, 14 and 19 days post CD117+ human AML cell lines injections. Treatment groups are indicated.

(C) Quantification of flow cytometry data showing the percentage of CD117+ GFP+human AML MOLM14 cells in the bone marrow of mice at termination of the experiment (day 19).

(D) Representative FACS plots showing the MOLM14 AML cells (identified as human CD45+GFP+ cells) in the bone marrow, peripheral blood and spleen of mice at termination of the experiment (day 19). Cells are represented as percentage of total live nucleated cells.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the invention, a bispecific antibody comprising at least a first binding domain and a second binding domain, wherein said first binding domain binds to an organic fluorophore and wherein said second binding domain binds to CD3.

Such new constructs are called “Universal T cell engaging and activating bispecific antibodies”, abbreviated UniTEA herein.

As used herein, the term “organic fluorophore” relates to organic small molecules that have the capacity to re-emit light upon light excitation. Organic fluorophores, as disclosed herein, have a maximum molecular that does not exceed 2500 Daltons. The term “organic fluorophore” excludes fluorescent proteins like green fluorescent protein (GFP), which has a molecular weight of 27 kDa and several phycobiliproteins, which have molecular weights of about 240 kDa.

In some embodiments, the organic fluorophore belongs to at least one of the groups selected from the list consisting of
Xanthene derivatives, like e.g. fluorescein, rhodamine, Oregon green, eosin, and Texas red
Cyanine derivatives, like e.g. cyanine, indocarbocyanine, oxacarbocyanine, thiacarbocyanine, and merocyanine
Squaraine derivatives and ring-substituted squaraines, including Seta and Square dyes Squaraine rotaxane derivatives, like e.g. See Tau dyes
Naphthalene derivatives (dansyl and prodan derivatives)
Coumarin derivatives
Oxadiazole derivatives, like e.g. pyridyloxazole, nitrobenzoxadiazole and benzoxadiazole
Anthracene derivatives, like e.g. anthraquinones, including DRAQ5, DRAQ7 and CyTRAK Orange
Pyrene derivatives, like e.g. cascade blue, etc.
Oxazine derivatives, like e.g. Nile red, Nile blue, cresyl violet, oxazine 170, etc.
Acridine derivatives, like e.g. proflavin, acridine orange, acridine yellow, etc.
Arylmethine derivatives, like e.g. auramine, crystal violet, malachite green
Tetrapyrrole derivatives, like e.g. porphin, phthalocyanine, bilirubin, and/or
Dipyrromethene derivatives, like e.g. BODIPY, aza-BODIPY

The inventors have found, surprisingly, that such constructs can be used, together with a second binding agent that is labelled with a corresponding fluorophore (also called “adaptor” herein), to bring T cell engagement therapy to almost every conceivable target in a patient's body.

Hence, a modular system is provided which facilitates the application of T cell engagement therapy to targets that have so far not been available for this approach. Further, such system may facilitate and accelerate regulatory approval for T cell engagement therapies against new targets.

The resulting UniTEA and fluorophore-tagged adaptor complex facilitates formation of a cytolytic synapse between the CD3 expressing T-cells and target cells, leading to effective target cell killing.

The UniTEA can be provided as an off-the-shelf GMP-manufactured biopharmaceutical and can be then used in combination with clinically approved therapeutic antibodies or other target-binders (called “adaptors” herein), which will be minimally modified (fluorophore-tagged) in order to make them suitable adaptors to redirect T-cell cytotoxicity to the respective target of interest.

Since organic fluorophores can be linked to a wide range of adaptors, T-cells can be redirected universally to any target cell of choice, provided there is an available clinical-grade bridge. Moreover, the antibodies (or peptides, small molecules, or any other antigen-binder) that would serve as appropriate adaptors will have their functionality enhanced due to UniTEs recruiting T cells and redirecting them via the adaptors.

The UniTEA modular system would allow an on-demand T-cell activation, which would be elicited only when the UniTEA is present concomitantly to the fluorophore-tagged, target bound adaptor, while no T-cell cytotoxic activity can be elicited in the absence of the UniTEA even when fluorophore-tagged bridges may persist in the body. Therefore, UniTEAs short half-life (˜30 min) exerts a precise control over endogenous T cell activity allowing for a fine tuning of the effects by dosing.

Antigen negative relapse or tumor heterogeneity would be addressed by exploiting combinatorial strategies that can be further personalized for each patient or disease, respectively. This system therefore allows existing clinically used target-binding molecules to be utilized in combinations or serially, allowing to target a wider and more heterogeneous range of tumor cell antigens (hematologic and non-hematologic tumors) overcoming the limitations of mono-specific tumor antigen targeting approaches.

Compared to the manufacturing costs of novel single- or multi-valent CAR-T cells as well as modular CAR-T cells which all require a dedicated GMP facility to genetically engineer patients' T-cells, the investment for developing a protein is sensibly lower. The advantages of generating a single universal bispecific protein supersede the production of de novo bispecific antibodies. The development of UniTE molecules would represent an alternative to the very demanding multivalent antibodies and CAR T-cells, while keeping the personalized advantages of a tailored therapeutical and the GMP production cost of a single recombinant protein.

According to one embodiment, said second binding domain binds to the c chain of CD3 and/or the γ chain of CD3.

The T cell receptor (TCR) complex is composed of ligand-binding subunits (TCRα and TCRβ) and signal transducing subunits (CD3γ, CD3δ, CD3ε and CD3ζ[CD247]) The ligand of the TCR consists of a peptide antigen bound to major histocompatibility complex (MHC) class I or class II molecules. Assembly studies, transfection and reconstitution experiments, and detergent dissociation studies suggest that the TCR complex components are organized as dimers [reviewed in. CD3ε forms non-covalently-bound alternate dimers with CD3γ and CD3δ, TCRα forms disulfide-linked dimers with TCRβ, and CD3ζ is expressed in the form of disulfide-linked homodimers.

T cells can be engaged by means of binding molecules capable of binding to CD3, in particular to CD3ε, the invariant signaling component of the T cell receptor complex. This takes advantage of the ability to harness polyclonal populations of cytotoxic T cells [CD8+, CD4+, including regulatory T cells (Tregs)] and is not dependent on MHC class I for antigen presentation. The monovalent binding of CD3 does not activate the TCR unless target cell binding is engaged, as will be discussed below, in a combination of the bispecific antibody and the labelled binding agent. In such way, engagement of T cells drives activation and resultant proliferation of the T cells which contributes to enhanced efficacy. Moreover. T cell engagement contributes to serial lysis which further enhances efficacy.

According to one embodiment of the invention, the bispecific antibody is in the format of a full-length antibody or an antibody fragment, the latter retaining target binding properties.

Generally, a large number of formats for bispecifc antibodies has been evaluated. For a review, see, e.g., Spiess et al. (2015), the content of which is incorporated herein by reference. Bispecific antibodies are classified into five distinct structural groups:
(i) bispecific IgG (BsIgG)
(ii) IgG appended with an additional antigen-binding moiety
(iii) bispecific antibody fragments
(iv) bispecific fusion proteins and
(v) bispecific antibody conjugates

Further, it is often differentiated between
(i) asymmetric bispecific antibodies (heteropolymeric antibodies with different chains comprising binding moieties to different antigens) and
(ii) symmetric bispecific antibodies, (heteropolymeric or monomeric/single chain antibodies, where the different binding moieties to different antigens are on the same chain)

Generally, small formats, which are often in the monomeric/single chain configuration) have a short serum half life, which makes frequent or even constant dosing necessary, yet provides the option to interrupt treatment immediately upon occurrence of side effects, like cytokine storms.

Asymmetric bispecific antibodies often pose problems when it comes to post-translational pairing of the different chains In the simultaneous expression of four different antibody chains (heavy and light chain for each specificity) irregular pairing can occur, leading to 10 different products, out of which only one is the correct variant. These mispairing issues can however be tackled with different technological approaches (see Wang et al. 2018).

See FIG. 5 for an overview of the different formats. Each of these formats brings different properties and advantages and can hence be chosen as a preferred format, inter alia,
binding valency for each antigen,
geometry of antigen-binding sites, c
correctness of chain pairing,
pharmacokinetic half-life
and in some cases effector functions.

According to embodiments of the invention, the bispecific antibody is in the format of at least one selected from the group consisting of
DVD-Ig
Knobs in Holes
Crossmab
BiTE™
Nanobody
Diabody
DART™
TandAb™^{,} and/or
scDb-scFv

These formats are well known to the skilled artisan (see Spiess et al. (2015) for the definitions). The skilled artisan can use the variable domains and/or sets of CDRs as disclosed herein to create the above mentioned formats by routine measures.

According to one embodiment of the invention, the bispecific antibody is in the format of a BiTE.

The BiTE format essentially consists of two scFv formatted antibodies joined to one another by means of a peptide linker, usually a linker comprising the amino acid sequence GGGGs (“G4S”). The resulting single chain has a weight of about 55 kD. Said BiTE can have the following formats (with the dash between the variable domains representing optional linkers):
VH₁-VL₁-VL₂-VH₂
VL₁-VH₁-VH₂-VL₂
VL₁-VH₁-VL₂-VH₂
VH₁-VL₁-VH₂-VL₂

Because of its small size, the BiTE format is associated with a very rapid clearance profile in patients. Blinatumomab, the only clinically-approved BiTE on the market, is administered as 28-day continuous infusion, thanks to the use of an intravenous pump with constant flow. In such way, the opportunity exists to immediately suspend antibody administration if serious adverse events occur.

The scDb-scFv is similar to the BiTE format, only with the difference that the variable domains for the first or second target are duplicated, es e.g. shown in the following:
VH₁-VL₁-VH₁-VL₁-VL₂-VH₂
VL₁-VH₁-VL₁-VH₁-VH₂-VL₂
VL₁-VH₁-VL₁-VH₁-VL₂-VH₂
VH₁-VL₁-VH₁-VL₁-VH₂-VL₂

The TandAb (Tandem Diabody) format is for example as follows:

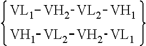

The DART format is a dimeric format where two chains are connected to one another by a cysteine linker:
Chain 1: VL₁-VH₂
Chain 2: VH₁-VL₂

Anticalin proteins are artificial proteins that are able to bind to antigens, either to proteins or to small molecules. They are not structurally related to antibodies, which makes them a type of antibody mimetic. Instead, they are derived from human lipocalins which are a family of naturally binding proteins. Anticalin proteins are being used in lieu of monoclonal antibodies, but are about eight times smaller with a size of about 180 amino acids and a mass of about 20 kDa.

According to one embodiment, said first binding domain of the bispecific antibody binds to Fluorescein, preferably Fluorescein when bound to a protein (conjugated Fluorescein).

As used herein, the terms “Fluorescein”, “Fluorescein isothiocyanate” and “FITC” are being used interchangeably. Binding agents, including antibodies binding to Fluorescein, preferably Fluorescein when bound to a protein (conjugated Fluorescein), are widely available. While some embodiments will be discussed in the following, reference is also made to the internet resource Biocomepare, which lists >78000 Anti-FITC Antibody Products, with ab19224 (Abeam, Goat polyclonal IgG) and LS C34608 (LSBio, Monoclonal Mouse FITC Antibody (clone F4/1, IHC), to name only a few.

According to one embodiment, said first binding domain of the bispecific antibody which binds to Fluorescein or conjugated Fluorescein comprises the anticalin FluA (SEQ ID NO: 47), which is disclosed in Vopel et al. (2005), the content of which is incorporated herein by reference.

As discussed elsewhere herein, anticalin proteins are artificial proteins that are able to bind to antigens, either to proteins or to small molecules. They are derived from human lipocalins which are a family of naturally binding proteins.

Preferably, said first binding domain of the bispecific antibody which binds to Fluorescein or conjugated Fluorescein comprises an anticalin FluA that has a sequence identity of ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% to SEQ ID NO: 47.

In the following, embodiments will be described in which specific antibody sequences are referred to. In some embodiments, alternative CDR (complementarity determining regions) sequences will be disclosed. These embodiments relate to the same antibody, but only to different nomenclatures according to which the CDRs were defined.

As used herein, the term “CDR” or “complementarity determining region” is intended to mean the non-contiguous antigen combining sites found within the variable region of both heavy and light chain polypeptides. These particular regions have been described by Kabat et al. (1977), Kabat et al. (1991), Chothia et al. (1987) and MacCallum et al., (1996) where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. Note that this numbering may differ from the CDRs that acre actually disclosed in the enclosed sequence listing, because CDR definitions vary from case to case.

TABLE 1
CDR definitions

| | Kabat | Chothia | MacCallum |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

In one embodiment, the term “capable to bind to target X” is to be understood as meaning binding with a sufficient binding affinity. In such embodiment, the respective binding domain binds the target with a K_{D} of 10⁻⁴ or smaller. K_{D} is the equilibrium dissociation constant, a ratio of k_{off}/kₒₙ, between the antibody and its antigen. K_{D} and affinity are inversely related. The K_{D} value relates to the concentration of antibody (the amount of antibody needed for a particular experiment) and so the lower the K_{D} value (lower concentration) and thus the higher the affinity of the binding domain. The following table shows typical K_{D} ranges of monoclonal antibodies:

TABLE 2
K_{D} and Molar Values

| K_{D} value | Molar range |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

Acording to one embodiment, the first binding domain which binds to Fluorescein or conjugated Fluorescein
a) comprises a set of heavy chain/light chain complementarity determining regions (CDR) comprised in the heavy chain/light variable region sequence pair of one of the anti Fluorescein antibodies E2 or 4m5.3, as set forth in SEQ ID NOs 27 and 31, or SEQ ID NOs 36 and 40, respectively
b) comprises the set of heavy chain/light chain complementarity determining regions (CDR) comprising the following sequences
   HC CDR1 (SEQ ID NO 28)
   HC CDR2 (SEQ ID NO 29)
   HC CDR3 (SEQ ID NO 30)
   LC CDR1 (SEQ ID NO 32)
   LC CDR2 (SEQ ID NO 33), and
   LC CDR3 (SEQ ID NO 34)
c) comprises the set of heavy chain/light chain complementarity determining regions (CDR) comprising the following sequences
   HC CDR1 (SEQ ID NO 37)
   HC CDR2 (SEQ ID NO 38)
   HC CDR3 (SEQ ID NO 39)
   LC CDR1 (SEQ ID NO 41)
   LC CDR2 (SEQ ID NO 42), and
   LC CDR3 (SEQ ID NO 43)
d) comprises the heavy chain/light chain complementarity determining regions (CDR) of
b) or c), with the proviso that at least one of the CDRs has up to 3 amino acid substitutions relative to the respective SEQ ID NO 28-30, 32-34, 37-39 or 41-43, and/or
e) comprises the heavy chain/light chain complementarity determining regions (CDR) of b), with the proviso that at least one of the CDRs has a sequence identity of ≥66% to the respective SEQ ID NO 28-30, 32-34, 37-39 or 41-43,

wherein the CDRs are embedded in a suitable protein framework so as to be capable to bind to Fluorescein or conjugated Fluorescein.

The anti-fluorescein antibody E2 is disclosed in Vaughan et al. (1996), the content of which is incorporated herein by reference. The anti-fluorescein antibody 4m5.3 is disclosed in Boder et al. (2000), the content of which is incorporated herein by reference.

Preferably, and this applies to all CDR sets disclosed herein, at least one CDR has up to 2 amino acid substitutions, and more preferably up to 1 amino acid substitutions

Preferably, and this applies to all CDR sets disclosed herein, at least one of the CDRs has a sequence identity of ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% to the respective SEQ ID NO.

As used herein, the term “% sequence identity”, has to be understood as follows: Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm is integrated in the BLAST family of programs, e.g. BLAST or NBLAST program and FASTA. Sequences which are identical to other sequences to a certain extent can be identified by these programmes. Furthermore, programs available in the Wisconsin Sequence Analysis Package, version 9.1 for example the programs BESTFIT and GAP, may be used to determine the % identity between two polypeptide sequences. If herein reference is made to an amino acid sequence sharing a particular extent of sequence identity to a reference sequence, then said difference in sequence is preferably due to conservative amino acid substitutions. Preferably, such sequence retains the activity of the reference sequence, e.g. albeit maybe at a slower rate.

Preferably, and this applies to all CDR sets disclosed herein, at least one of the CDRs has been subject to CDR sequence modification, including
affinity maturation
reduction of immunogenicity

Affinity maturation in the process by which the affinity of a given antibody is increased in vitro. Like the natural counterpart, in vitro affinity maturation is based on the principles of mutation and selection. It has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range. For principles see Eylenstein et al. (2016), the content of which is incorporated herein by reference.

Humanized antibodies contain murine-sequence derived CDR regions that have been engrafted, along with any necessary framework back-mutations, into human sequence-derived V regions. Hence, the CDRs themselves can cause immunogenic reactions when the humanized antibody is administered to a patient. Methods of reducing immunogenicity caused by CDRs are disclosed in Harding et al. (2010), the content of which is incorporated herein by reference. According to one embodiment of the invention, the framework is a human VH/VL framework. VH stands for heavy chain variable domain of an IgG shaped antibody, while VL stands for light chain variable domain (kappa or lambda).

According to one embodiment of the invention, the first binding domain which binds to Fluorescein or conjugated Fluorescein comprises
a) the variable domains of one antibody selected from the group consisting of E2 or 4m5.3
b) the heavy chain/light chain variable domains (VD)
   HC VD (SEQ ID NO 27 or 36), and
   LC VD (SEQ ID NO 31 or 40)
c) the heavy chain/light chain variable domains (VD) of b), with the proviso that
   the HCVD has a sequence identity of ≥80% to the respective SEQ ID NO 27 or 36, and/or
   the LCDVD has a sequence identity of ≥80% to the respective SEQ ID NO 31 or 40
d) the heavy chain/light chain variable domains (VD) of b), with the proviso that at least one of the HCVD or LCVD has up to 10 amino acid substitutions relative to the respective SEQ ID NOs,

wherein said bispecific antibody is still capable to bind to Fluorescein or conjugated Fluorescein.

A “variable domain” when used in reference to an antibody or a heavy or light chain thereof is intended to mean the portion of an antibody which confers antigen binding onto the molecule and which is not the constant region. The term is intended to include functional fragments thereof which maintain some of all of the binding function of the whole variable region. Variable region binding fragments include, for example, functional fragments such as Fab, F(ab)₂, Fv, single chain Fv (scfv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Huston et al., (1993) or PlUckthun and Skerra (1990).

Preferably, and this applies to all variable domains disclosed herein, at least one of the variable domains has ≤9, ≤8, ≤7, ≤6, ≤5, ≤4, ≤3, ≤2, ≤1 amino acid substitutions relative to the respective SEQ ID NO.

Preferably, and this applies to all variable domains disclosed herein, at least one of the variable domains has a sequence identity of ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99 to the respective SEQ ID NO.

Preferably, and this applies to all variable domains disclosed herein, at least one amino acid substitution in any of the variable domain disclosed herein is a conservative amino acid substitution

A “conservative amino acid substitution” has a smaller effect on antibody function than a non-conservative substitution. Although there are many ways to classify amino acids, they are often sorted into six main groups on the basis of their structure and the general chemical characteristics of their R groups.

In one embodiment, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Thus, a predicted nonessential amino acid residue in a HR domain polypeptide, for example, is preferably replaced with another amino acid residue from the same side chain family or homologues across families (e.g. asparagine for aspartic acid, glutamine for glutamic acid). Conservative changes can further include substitution of chemically homologous non-natural amino acids (i.e. a synthetic non-natural hydrophobic amino acid in place of leucine, a synthetic non-natural aromatic amino acid in place of tryptophan).

According embodiments of the invention, the second binding domain which binds to CD3 comprises the CDRs and/or variable domains of an antibody selected from the group consisting of OKT3 or BlinCD3 as well as, UCHT-1 (Ceuppens et al, 1986), BMA031 (Borst et al, 1990) and 12F6 (Li et al, 2005).

The anti CD3 antibody OKT3 is disclosed in Horn et al. (2017) the content of which is incorporated herein by reference. The anti CD3 antibody BlinCD3 is a modified variant of OKT3.

According to one embodiment of the invention, the second binding domain which binds to CD3
a) comprises a set of heavy chain/light chain complementarity determining regions (CDR) comprised in the heavy chain/light variable region sequence pair of one of the anti CD3 antibodies BlinCD3 and OKT3, as set forth in SEQ ID NOs 1 and 8, or SEQ ID NOs 16 and 20, respectively
b) comprises the set of heavy chain/light chain complementarity determining regions (CDR) comprising the following sequences
   HC CDR1 (SEQ ID NO 2 or 5)
   HC CDR2 (SEQ ID NO 3 or 6)
   HC CDR3 (SEQ ID NO 4 or 7)
   LC CDR1 (SEQ ID NO 9 or 12)
   LC CDR2 (SEQ ID NO 10 or 13), and
   LC CDR3 (SEQ ID NO 11 or 14)
c) comprises the set of heavy chain/light chain complementarity determining regions (CDR) comprising the following sequences
   HC CDR1 (SEQ ID NO 17)
   HC CDR2 (SEQ ID NO 18)
   HC CDR3 (SEQ ID NO 19)
   LC CDR1 (SEQ ID NO 21)
   LC CDR2 (SEQ ID NO 22), and
   LC CDR3 (SEQ ID NO 23)
d) comprises the heavy chain/light chain complementarity determining regions (CDR) of
b) or c), with the proviso that at least one of the CDRs has up to 3 amino acid substitutions relative to the respective SEQ ID NO 2-4, 9-11, 17-19 or 21-23, and/or
e) comprises the heavy chain/light chain complementarity determining regions (CDR) of b), with the proviso that at least one of the CDRs has a sequence identity of ≥66% to the respective SEQ ID NO 2-4, 9-11, 17-19 or 21-23,

wherein the CDRs are embedded in a suitable protein framework so as to be capable to bind to CD3.

With regard to item b), two different sets of CDRs are disclosed, because the counting of CDRs may vary from one counting method to the other (e.g., Kabat, Chothia or MacCallum).

According to one embodiment of the invention, the second binding domain which binds to CD3 comprises
a) the variable domains of one antibody selected from the group consisting of OKT3 and BlinCD3
b) the heavy chain/light chain variable domains (VD)
   HC VD (SEQ ID NO 1 or 16), and
   LC VD (SEQ ID NO 8 or 20)
c) the heavy chain/light chain variable domains (VD) of b), with the proviso that
   the HCVD has a sequence identity of ≥80% to the respective SEQ ID NO 1 or 16, and/or
   the LCDVD has a sequence identity of ≥80% to the respective SEQ ID NO 8 or 20
d) the heavy chain/light chain variable domains (VD) of b), with the proviso that at least one of the HCVD or LCVD has up to 10 amino acid substitutions relative to the respective SEQ ID NOs,

wherein said bispecific antibody is still capable to bind to CD3.

According to one embodiment, the framework is a human VH/VL framework. According to one embodiment, at least one amino acid substitution is a conservative amino acid substitution.

According to one embodiment of the bispecific antibody, the second binding domain which binds to CD3
a) comprises a scFv-like sequence as set forth in any one of SEQ ID NOs 15 or 24, or
b) comprises an amino acid sequence that has a sequence identity of ≥80% to any one of SEQ ID NOs 15 or 24.

According to one embodiment of the bispecific antibody, the first binding domain which binds to Fluorescein or conjugated Fluorescein
a) comprises a scFv-like sequence as set forth in any one of SEQ ID NOs 35 or 44, or
b) comprises an amino acid sequence that has a sequence identity of ≥80% to any one of SEQ ID NOs 35 or 44.

According to one embodiment of the bispecific antibody, the first binding domain which binds to Fluorescein or conjugated Fluorescein
a) comprises at least one of the sequences selected from SEQ ID NOs 48-50, or
b) comprises an amino acid sequence that has a sequence identity of ≥80% to SEQ ID NOs 48-50,

with optionally a C terminal cysteine residue and/or a His tag removed or in place.

According to one embodiment of the bispecific antibody, the agent has at least one of
target binding affinity of ≥50% to CD3, and measured by SPR, and/or
target binding affinity of ≥50% to Fluorescein or conjugated Fluorescein, and measured by SPR,

compared to that of the bispecific antibody according to the above description.

According to another aspect of the invention, a bispecific antibody is provided which competes for binding to Fluorescein or conjugated Fluorescein, and/or CD3, respectively, with the bispecific antibody according to the above description.

According to another aspect of the invention, a nucleic acid is provided which encodes for a binding agent according to the above description. Such nuclei acid can be an mRNA, a cDNA, a DNA or a genomic DNA, which can comprised non coding stretches (Introns). Due to the degeneracy of the gentic code, a large number of different nucleic acid sequences can enclose for the same binding agent. However, based on the sequence information of the binding agent the skilled person can determine or identify one or more such nucleic acid molecules.

According to another aspect of the invention, a combination is provided comprising
a) the bispecific antibody according to the above description, and
b) a labelled binding agent that binds specifically to a target antigen,

wherein the labelled binding agent is labelled with an organic fluorophore that is specifically detected and/or bound by the first binding domain of the bispecific antibody according to the above description.

According to another aspect of the invention, a dosage regimen is provided, comprising administration to a patient, consecutively or concomitantly. of
a) the bispecific antibody according to the above description, and
b) a labelled binding agent that binds specifically to a target antigen,

wherein the labelled binding agent is labelled with an organic fluorophore that is specifically detected and/or bound by the first binding domain of the bispecific antibody according to the above description.

The labelled binding agent is also called “adaptor molecule” or “adaptor” herein, as it provides target specificity to the combination. Technically, the labelled binding agent can be chosen from large libraries of binding agents, including antibodies, that are specific to every conceivable target antigen.

In one embodiment, the target antigen is an antigen that is presented by tumor cells of a hematological tumor. In one embodiment, the target antigen is an antigen that is presented by tumor cells of a solid tumor.

The advantages of using an organic fluorophore in the adaptor molecule are manifold:
a) for many organic fluorophores commercially available antibodies with high affinity exist that can be used in the bispecific antibody
b) organic fluorophores are relatively small (fluorescein has e.g. a molecular weight of about 390 Da)
c) organic fluorophores are non-immunogenic and non-toxic (fluorescein is being used in eye drops for example)
d) a binding agent that is labelled with an organic fluorophore can be monitored e.g. in preclinical studies
e) proven chemistry exists to stably conjugate organic fluorophores to binding agents like e.g. antibodies, avoiding dissociation of the organic fluorophore from the binding agent in the bloodstream (as could happen for peptide labels).

As regards the bispecific antibody in the said combination, the same embodiments apply as disclosed above for the bispecific antibody alone.

According to one embodiment of the combination or dosage regimen according to the above description, the bispecific antibody according and the labelled binding agent are administered individually, or are first mixed with one another and then administered.

According to one embodiment of the combination or dosage regimen according to the above description, the organic fluorophore in the labelled binding agent is fluorescein.

As used herein, the terms “Fluorescein”, “Fluorescein isothiocyanate” and “FITC” are being used interchangeably.

According to one embodiment of the combination or dosage regimen according to the above description, the labelled binding agent is site-specifically labelled with the organic fluorophore.

In such way, it can be assured that
a) a constant stochimetry ratio between binding agent and organic fluorophore can be achieved, which is crucial for a reproducible dosage of the combination of bispecific antibody and labelled binding agent.
b) the labelling does not interfere with the binding of the labelled bispecific agent to its target.

In one embodiment, a site specific labeling with fluorescein (“FITCylation” can be obtained by the use of (i) fluorescein-5-maleimide (5-MF), and (ii) a proteinaceous binding agent—like an antibody, fragment or derivative thereof, that has a C-terminal cysteine.

Such approach is disclosed in the experimental section herein, and was for example described in Pellegrino et al. 2020), the content of which is incorporated herein by reference for enablement purposes.

According to one embodiment of the combination or dosage regimen according to the above description, the labelled binding agent comprises an antibody, or a target binding fragment or derivative thereof.

In a preferred embodiment, the labelled binding agent is at least one selected from the group consisting of an
antibody,
antibody-based binding protein
modified antibody format retaining target binding capacity,
antibody derivative or fragment retaining target binding capacity, and/or
antibody mimetic.

As used herein, the term “binding agent” is meant to define an entity, an agent or a molecule that has affinity to a given target, e.g., a receptor, a cell surface protein, a cytokine or the like. Such Binding agent may optionally block or dampen agonist-mediated responses, or inhibit receptor-agonist interaction. Most importantly, however, the binding agent may serve as a shuttle to deliver a payload to a specific site, which is defined by the target recognized by said binding agent. Thus, a binding agent targeting, for instance, but not limited to a receptor, delivers its payload to a site which is characterized by abundance of said receptor.

Binding agent s include, but are not limited to, antibodies, antibody fragments, antibody-based binding proteins, antibody mimetics, receptors, soluble decoy receptors, scaffold proteins with affinity for a given target and ligands of receptors.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid antibody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgGl, IgG2, IgG3, IgG4, IgA1, and IgA2) and allotype.

An “antibody-based binding protein”, as used herein, may represent any protein that contains at least one antibody-derived V_{H}, V_{L}, or C_{H} immunoglobulin domain in the context of other non-immunoglobulin, or non-antibody derived components. Such antibody-based proteins include, but are not limited to (i) F_{c}-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (V_{L}), variable heavy (V_{H}), constant light (C_{L}) and constant heavy 1 (C_{H}I) domains; (ii) a F(ab′)2 fragment, which is a bivalent fragment comprising two Fab fragments linked by a disulfide bridge at the hinge region; (iii) a heavy chain portion of a F_{ab} (Fa) fragment, which consists of the V_{H} and C_{H}I domains; (iv) a variable fragment (Fᵥ) fragment, which consists of the V_{L} and V_{H} domains of a single arm of an antibody, (v) a domain antibody (dAb) fragment, which comprises a single variable domain; (vi) an isolated complementarity determining region (CDR); (vii) a single chain Fᵥ Fragment (scFᵥ); (viii) a diabody, which is a bivalent, bispecific antibody in which V_{H} and V_{L} domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem Fᵥ segments (V_{H}-C_{H}1-V_{H}-C_{H}1) which, together with complementarity light chain polypeptides, form a pair of antigen binding regions; (X) Dual-Variable Domain Immunoglobulin (xI) other non-full length portions of immunoglobulin heavy and/or light chains, or mutants, variants, or derivatives thereof, alone or in any combination.

The term “modified antibody format”, as used herein, encompasses antibody-drug-conjugates, Polyalkylene oxide-modified scFv, Monobodies, Diabodies, Camelid Antibodies, Domain Antibodies, bi- or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerised constructs comprising CH3+VL+VH, and the like.

The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, nucleic acid aptamers, artificial antibodies produced by molecular imprinting of polymers, peptide libraries from bacterial genomes, SH-3 domains, stradobodies, “A domains” of membrane receptors stabilised by disulfide bonds and Ca2+, CTLA4-based compounds, and Fyn SH3.

In one embodiment, the labelled binding agent is a scfV fragment, for example having one of the following structures:
VL₁-VH₁
VH₁-VL₁

In one embodiment, the labelled binding agent is a diabody, for example having one of the following structures:
VL₁-VH₁-VL₁-VH₁
VH₁-VL₁-VH₁-VL₁

According to embodiments of the combination or dosage regimen according to the above description, the labelled binding agent binds specifically to a target antigen selected from the group consisting of CD117 and CAIX.

Preferably, CD117/c-KIT is human CD117/c-KIT as e.g. disclosed under UniProt identifier P10721. Mast/stem cell growth factor receptor (SCFR), also known as proto-oncogene c-KIT or tyrosine-protein kinase KIT or CD117, is a receptor tyrosine kinase protein that in humans is encoded by the KIT gene. Multiple transcript variants encoding different isoforms have been found for this gene. KIT was first described by the German biochemist Axel Ullrich in 1987 as the cellular homolog of the feline sarcoma viral oncogene v-kit.

CD117 is a cytokine receptor expressed on the surface of hematopoietic stem cells as well as other cell types. Altered forms of this receptor may be associated with some types of cancer. CD117 is a receptor tyrosine kinase type III, which binds to stem cell factor (a substance that causes certain types of cells to grow), also known as “steel factor” or “c-kit ligand”. When this receptor binds to stem cell factor (SCF) it forms a dimer that activates its intrinsic tyrosine kinase activity, that in turn phosphorylates and activates signal transduction molecules that propagate the signal in the cell. After activation, the receptor is ubiquitinated to mark it for transport to a lysosome and eventual destruction. Signaling through CD117 plays a role in cell survival, proliferation, and differentiation. For instance, CD117 signaling is required for melanocyte survival, and it is also involved in haematopoiesis and gametogenesis.

Like other members of the receptor tyrosine kinase III family, CD117 consists of an extracellular domain, a transmembrane domain, a juxtamembrane domain, and an intracellular tyrosine kinase domain. The extracellular domain is composed of five immunoglobulin-like domains, and the protein kinase domain is interrupted by a hydrophilic insert sequence of about 80 amino acids. The ligand stem cell factor binds via the second and third immmunoglobulin domains.

Cluster of differentiation (CD) molecules are markers on the cell surface, as recognized by specific sets of antibodies, used to identify the cell type, stage of differentiation and activity of a cell. CD117 is an important cell surface marker used to identify certain types of hematopoietic (blood) progenitors in the bone marrow. To be specific, hematopoietic stem cells (HSC), multipotent progenitors (MPP), and common myeloid progenitors (CMP) express high levels of CD117. Common lymphoid progenitors (CLP) express low surface levels of CD117. CD117 also identifies the earliest thymocyte progenitors in the thymus—early T lineage progenitors (ETP/DN1) and DN2 thymocytes express high levels of c-Kit. It is also a marker for mouse prostate stem cells. In addition, mast cells, melanocytes in the skin, and interstitial cells of Cajal in the digestive tract express CD117. In humans, expression of c-kit in helper-like innate lymphoid cells (ILCs) which lack the expression of CRTH2 (CD294) is used to mark the ILC3 population.

In humans, CD117/c-Kit is inter alia present in hematopoietic cells and plays a crucial role in early stages of hematopoiesis, but is also expressed in AML blasts

Preferably CAIX (Carbonic anhydrase IX, also called CA09) is human CAIX (UniProt identifier: A0A087WYS4. Carbonic anhydrase IX (CAIX) is a suitable target for various anticancer strategies. It is a cell surface protein that is present in human tumors, but not in the corresponding normal tissues. Expression of CAIX is induced by hypoxia and correlates with cancer prognosis in many tumor types. Moreover, CAIX is functionally implicated in cancer progression as a pro-survival factor protecting cancer cells against hypoxia and acidosis via its capability to regulate pH and cell adhesion.

Cancer-related distribution of CAIX allows for targeting cancer cells by antibodies binding to its extracellular domain, whereas functional involvement of CAIX opens the possibility to hit cancer cells by blocking their adaptation to physiologic stresses via inhibition of CAIX enzyme activity. The latter strategy is recently receiving considerable attention and great efforts are made to produce CAIX-selective inhibitor derivatives with anticancer effects.

Targeting CAIX-expressing cells by immunotherapy has reached clinical trials and is close to application in treatment of renal cell carcinoma patients. Nevertheless, development and characterization of new CAIX-specific antibodies is still ongoing.

According to one embodiment of the combination or dosage regimen according to the above description, the labelled binding agent that binds to CD117
a) comprises a set of heavy chain/light chain complementarity determining regions (CDR) comprised in the heavy chain/light variable region sequence pair of the anti CD117 antibody 79D, as set forth in SEQ ID NOs 52 and 59,
b) comprises the set of heavy chain/light chain complementarity determining regions (CDR) comprising the following sequences
   HC CDR1 (SEQ ID NO 53 or 56)
   HC CDR2 (SEQ ID NO 54 or 57)
   HC CDR3 (SEQ ID NO 55 or 58)
   LC CDR1 (SEQ ID NO 60 or 63)
   LC CDR2 (SEQ ID NO 61 or 64), and
   LC CDR3 (SEQ ID NO 62 or 65)
c) comprises the heavy chain/light chain complementarity determining regions (CDR) of b) or c), with the proviso that at least one of the CDRs has up to 3 amino acid substitutions relative to the respective SEQ ID NO 53-55, 60-62, 56-57 or 63-65, and/or
d) comprises the heavy chain/light chain complementarity determining regions (CDR) of b), with the proviso that at least one of the CDRs has a sequence identity of ≥66% to the respective SEQ ID NO 53-55, 60-62, 56-57 or 63-65,

wherein the CDRs are embedded in a suitable protein framework so as to be capable to bind to CD117

The anti CD117 antibody 79D is disclosed in X, the content of which is incorporated herein by reference.

With regard to item b), two different sets of CDRs are disclosed, because the counting of CDRs may vary from one counting method to the other (e.g., Kabat, Chothia or MacCallum).

According to one embodiment of the combination or dosage regimen according to the above description, the labelled binding agent that binds to CD117 comprises
a) the variable domains of the antibody 79D
b) the heavy chain/light chain variable domains (VD)
   HC VD (SEQ ID NO 52), and
   LC VD (SEQ ID NO 59)
c) the heavy chain/light chain variable domains (VD) of b), with the proviso that
   the HCVD has a sequence identity of ≥80% to the respective SEQ ID NO 52, and/or
   the LCDVD has a sequence identity of ≥80% to the respective SEQ ID NO 59
d) the heavy chain/light chain variable domains (VD) of b), with the proviso that at least one of the HCVD or LCVD has up to 10 amino acid substitutions relative to the respective SEQ ID NOs,

wherein said labelled binding agent is still capable to bind to CD117.

With regard to the different alternatives disclosed, the same embodiments apply as with regard to the bispecific antibody disclosed elsewhere herein, in particular regarding the homology ranges and the conservative amino acid substitutions.

With regard to the different alternatives disclosed, the same embodiments apply as with regard to the bispecific antibody disclosed elsewhere herein, in particular regarding the homology ranges and the conservative amino acid substitutions

According to another aspect of the invention, a pharmaceutical composition comprising the combination according the above description is provided, which optionally comprises one or more pharmaceutically acceptable excipients.

According to one aspect of the invention, the combination or dosage regimen or pharmaceutical composition according to the above description is provided for use (in the manufacture of a medicament) in the treatment of a human or animal subject
being diagnosed for,
suffering from or
being at risk of

developing a neoplastic disease, or for the prevention of such condition.

The above language with the term “in the manufacture of a medicament” in brackets, is meant to provide disclosure for both the so-called Swiss type claim language (where the brackets are deemed removed) and the claim language acceptable, e.g., under EPC 2000 (where the content between the brackets is deemed removed).

In several embodiments thereof, the bispecific antibody (a) and the labelled binding agent (b) are administered
consecutively, with a) before b) or b) before a), or
concomitantly.

In preferred embodiments, such neoplastic disease is a solid tumor or a haematological tumor. The examples provided in the present specification provided enabling support for both fields of application.

According to one aspect of the invention, a method for treating or preventing a neoplastic disease is provided, comprising administering to a subject in need thereof an effective amount of the combination or pharmaceutical composition according to the above description, or subjecting said patient to dosage regimen according to the above description.

In several embodiments thereof, the bispecific antibody (a) and the labelled binding agent (b) are administered
consecutively, with a) before b) or b) before a), or
concomitantly.

According to one aspect of the invention, a therapeutic kit of parts is provided, comprising:
a) the combination or pharmaceutical composition according to the above description
b) an apparatus for administering the combination or composition, and
c) optionally, instructions for use.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Materials and Methods

### Cell Lines

NALM-6 (ACC 128, DSMZ) and MOLM-14 (ACC 777, DSMZ) cells were cultured in RPMI-1640 supplemented with 20% FBS and 1% penicillin/streptomycin. Cell lines were expanded and stored as cryopreserved aliquots in liquid nitrogen. Cells were grown according the supplier's protocol and kept in culture for no longer than 2 weeks. Authentication of the cell line also included check of post-freeze viability, growth properties, morphology, test for mycoplasma contamination, isoenzyme assay, and sterility test were performed by the cell bank before shipment. MOLM-14 cells were lentivirally transduced and subsequently sorted to homogeneously express CD117 on their surface (Myburgh et al. 2020).

### Isolation and Purification of T-Cells

Healthy donors' buffy coats were acquired from the Zurich blood donation service (Blutspende Zurich, Zurich, Switzerland). Peripheral blood mononuclear cells (PBMCs) were isolated by density gradient centrifugation on Ficoll Paque Plus (GE Healthcare) according to the manufacturer's instructions. T-cells were then negatively isolated with EasySep™ beads (Human T cell isolation kit, STEMCELL Technologies). Purified T-cell samples were re-suspended in 90% FCS supplemented with 10% DMSO and cryo-preserved at −80° C. and subsequently in liquid nitrogen.

### Cloning of UniTE(4m5.3/BlinCD3), UniTE(E2/BlinCD3) and UniTE(FluA/BlinCD3)

The sequences of a humanized version of the anti-human CD3 antibody OKT3, called Blin CD3 (SEQ ID NOs 1-14) and the sequences of the anti-fluorescein murine 4m5.3 (SEQ ID NOs 36-34) or human E2 antibody (27.35) (disclosed in Boder et al. (2000) and Vaughan et al. (1996)), or anticalin™ FluA (disclosed in Vopel et al. (2005) (SEQ ID NO 47) were genetically assembled by PCR in the following formats to obtain the UniTEs 4m5.3/BlinCD3, FluA/BlinCD3 and E2/BlinCD3 (lower row shows the SEQ ID NOs):

| VL | | VH | | | | | | |
|---|---|---|---|---|---|---|---|---|
| (anti- | | (anti- | | VH | | VL | | |
| FITC) | Linker15aa | FITC) | Linker5aa | (BlinCD3) | Linker18aa | (BlinCD3 | 6xHis | Total |
| 40 | 45 | 36 | 67 | 1 | 26 | 8 | HHHHHH | 49 |

| FluA | Linker 5aa | VH (BlinCD3 | Linker 18aa | VL (BlinCD3 | 6xHis | Total |
|---|---|---|---|---|---|---|
| 47 | 67 | 1 | 26 | 8 | HHHHHH | 50 |

| VL | | VH | | | | | | |
|---|---|---|---|---|---|---|---|---|
| (anti- | | (anti- | | VH | | VL | | |
| FITC) | Linker15aa | FITC) | Linker5aa | (BlinCD3 | Linker18aa | (BlinCD3 | 6xHis | Total |
| 27 | 45 | 31 | 67 | 1 | 26 | 8 | HHHHHH | 48 |

And cloned into the mammalian expression vector pcDNA3.1(+) (Invitrogen).

### Cloning of Antibodies That to Be Used as Adaptors

Two different labelled binding agents (“adaptors”) were created, both of which are antibodies. One adaptor binds CD117, which, as discussed elsewhere herein, is a target expressed on blood cells and hematopoietic cells. It hence stands representative for haematological cancers.

The other adaptor binds to CAIX (Carbonic anhydrase IX, also called CA09), which as discussed elsewhere herein, is a cell surface protein that is present in human tumors, but not in the corresponding normal tissues. It hence stands representative for solid tumors.

The anti CD117 antibody is described in Reshetnyak et al. (2013), and bears the clonal name antibody 79D. It comprises the heavy chain/light chain variable domain HC VD (SEQ ID NO 52), and LC VD (SEQ ID NO 59).

The anti CAIX antibody is described in Cazzamalli et al (2018), and bears the clonal name antibody XE114. It comprises the heavy chain/light chain variable domain HC VD (SEQ ID NO 68), and LC VD (SEQ ID NO 72).

### Anti-CD117 Antibody

Sequences of the variable region of the light and heavy chains of the anti-CD117 antibody were cloned in a diabody (Db) format (VL-Linker 5aa-VH) with a C-terminal poly-histidine tag into pcDNA3.1(+). An additional C-terminal cysteine was added for site-selective conjugations.

### Anti-CAIX Antibody

The anti CAIX antibody was cloned and prepared as an IgG as described in Cazzamalli et al (2018), the content of which is incorporated herein by reference.

### Expression of Antibodies

The antibodies were produced by transient gene expression using polyethyleneimine in CHO-S cells (Invitrogen) following standard protocols⁸. The proteins were purified by Ni-NTA agarose resin (ThermoFisher) and analyzed using SDS-PAGE and size exclusion chromatography (Superdex 200 Increase, 10/300, GE Healthcare).

### Conjugation of Anti-CD117 and Anti-CAIX to Fluorescein

Antibody solutions in PBS (1 mg/ml) were reduced with 50 eq. of DTT (1,4-Dithio-DL-threitol) in PBS. After reduction, the protein solution was washed three times with PBS using Vivaspin centrifugation column (Sartorius) to dilute the DTT. Fluorescein-5-maleimide (Thermo Scientific) was dissolved in DMSO anhydrous to obtain a final concentration of 10% (v/v) when added to the reduced protein. Antibodies were treated with 50 eq. per cysteine of fluorescein, and homogeneous conjugates with single modification within the light chain was observed by LC-MS after 1 hr. Conjugated proteins were purified by PD-10 desalting columns (GE Healthcare) and eluted in Acetate Buffer, pH 5. Aliquots were snap-frozen in liquid nitrogen and stored at −80° C. until further use.

### In Vitro UniTE-Mediated T-Cell Killing of CD19⁺ Tumor Cells

Healthy donor's T cells were thawed 1 day before the assay and cultured overnight in advanced RPMI supplemented with lx Glutamax (Gibco), 10% FBS and 1% penicillin/streptomycin (T cell medium). T cells and CD19+B acute lymphoblastic leukemia (B-ALL) NALM-6 cells were co-incubated at 10:1 ratio (E:T=10:1) in presence of 10 nM of a commercially available FITC-anti-human CD19 mouse IgG1 (Biolegend; clone HIB19). UniTE(4m5.3/BlinCD3) and UniTE(FluA/BlinCD3) were compared with the clinically approved (anti-CD19/BlinCD3) bispecific T cell engager (BiTE™) Blinatumomab (Blycinto™) at the same concentrations (100 nM and 10 nM). The specific lysis of target cells was analyzed by flow cytometry using LSR II Fortessa cell analyzer (BD Biosciences) and quantified using the following formula: (1-number of alive target cells/number of alive target cells without antibody)*100.

### In vitro UniTE-Mediated T-Cell Killing of CD117⁺ Tumor Cells

T cells were thawed 1 day before the assay and cultured overnight in T cell medium. T cells and acute myeloid leukemia (AML) MOLM-14 cells (transduced to express CD117 on the surface) were co-cultured in T cell medium (E:T =1:1) in presence of 10 nM of anti CD117 diabody site-specifically conjugated with fluorescein. UniTE(4m5.3/BlinCD3) killing was compared with an anti-CD117/BlinCD3 bispecific T cell engager at equimolar concentrations (100 nM and 1 nM) and with direct anti-CD117 CAR-T cells¹, both bearing the scFv from the same clone of the bridging Db. The specific lysis of target cells was analyzed as previously described.

In Vitro UniTEA-Mediated T-Cell Killing of CAIX⁺ Tumor Cells

T cells were thawed 1 day before the assay and cultured overnight in T cell medium. CAIX⁺ renal cell carcinoma SK-RC-52 cells were harvested 1 day before the assay and membrane stained using PKH26 Red Fluorescent Cell Linker Kit for General Membrane (Sigma-Aldrich) according to manufacturer instructions. Stained SK-RC-52 cells were seeded in a 96 well plate and incubated overnight. The next day, SK-RC-52 cells and T cells were co-cultured in T cell medium (E:T=10:1) in presence of 10 nM site-specifically fluoresceinated anti-CAIX IgG and different concentration of 4m5.3/BlinCD3, FluA/BlinCD3 and E2/BlinCD3 UniTEAs (1, 10 and 100 nM). After 24 hrs, the specific lysis of target cells was analyzed as previously described. Background killing after co-culturing T- and tumor cells alone or by adding only 10 nM adaptor molecule was also analyzed.

### In Vivo Stability of Fluorescein Molecules on Antibodies

8-week old C57BL/6J mice were i. v. injected with 50 ug of anti-murine CD19-FITC antibodies (Biolegend; clone 1D3). Murine B cells were analyzed by flow cytometry from peripheral blood at 4 hrs, 1 day, 4 days and 8 days post injection. The peripheral blood was lysed for 10min with RBC lysis reagent (Biolegend) and washed 1× with PBS (Invitrogen). Each sample was split in 2 and stained with only anti-mouse CD20 (Biolegend; clone SA271G2) or with anti-mouse CD20 (Biolegend; clone SA271G2) and anti-mouse CD19-FITC (Biolegend; clone 1D3). Samples were stained in FACS buffer (PBS with 2% FBS and 2mM EDTA) and washed twice after incubation prior to FACS analysis.

### Statistical Analysis

Data are reported as mean±standard deviation (SD) unless otherwise noted. Statistical analysis was performed GraphPad Prism 8.0 software. FlowJo software (v10.0.7, FlowJo) was used for data analysis and presentation.

### Results

### Production of Various UniTEA Formats

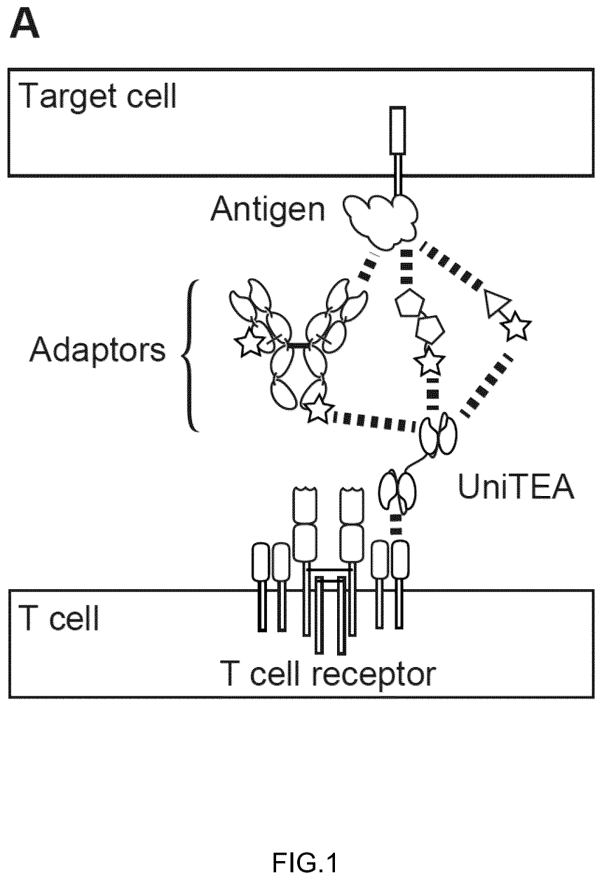

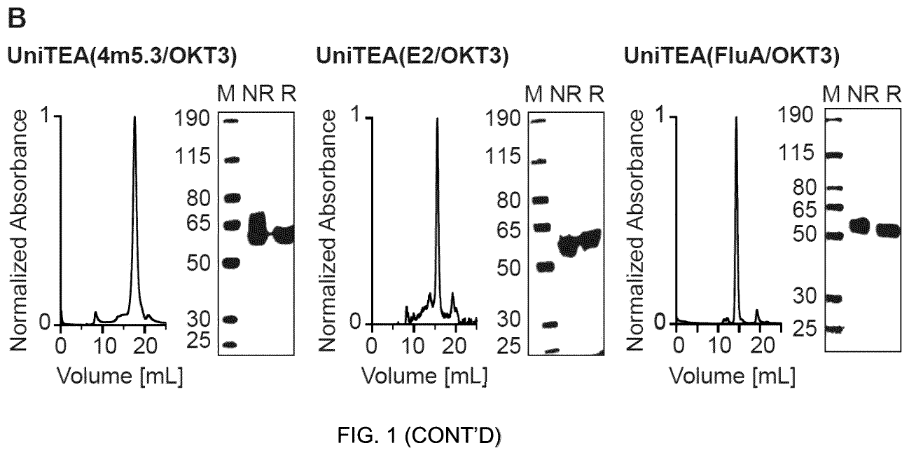

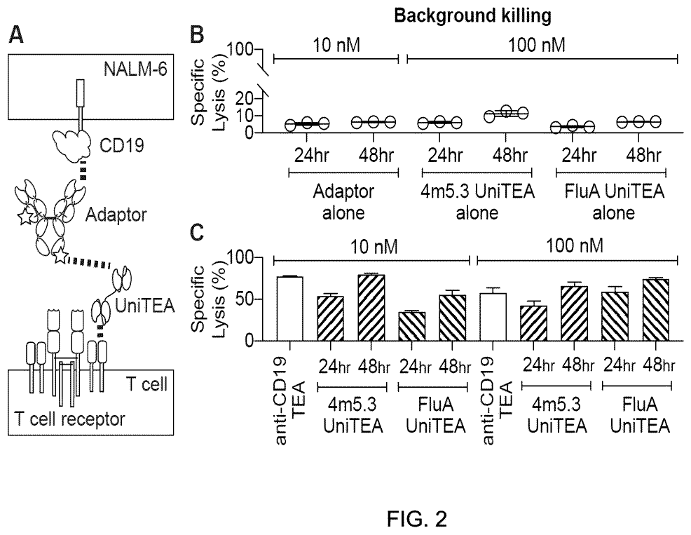

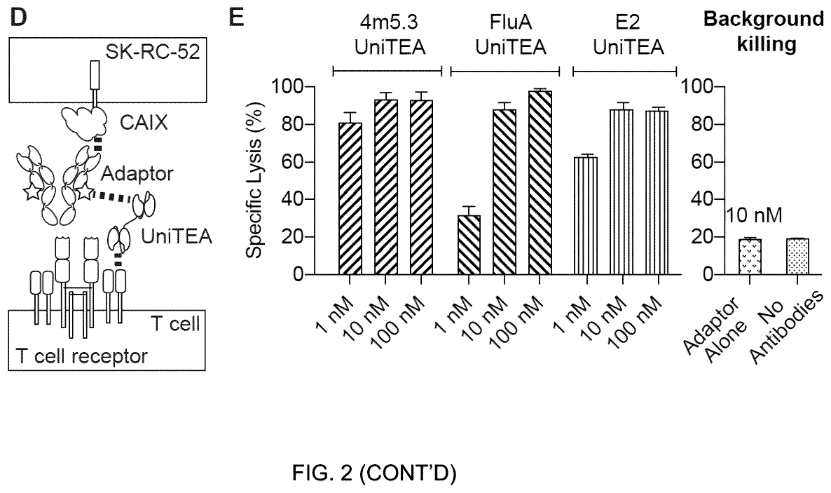

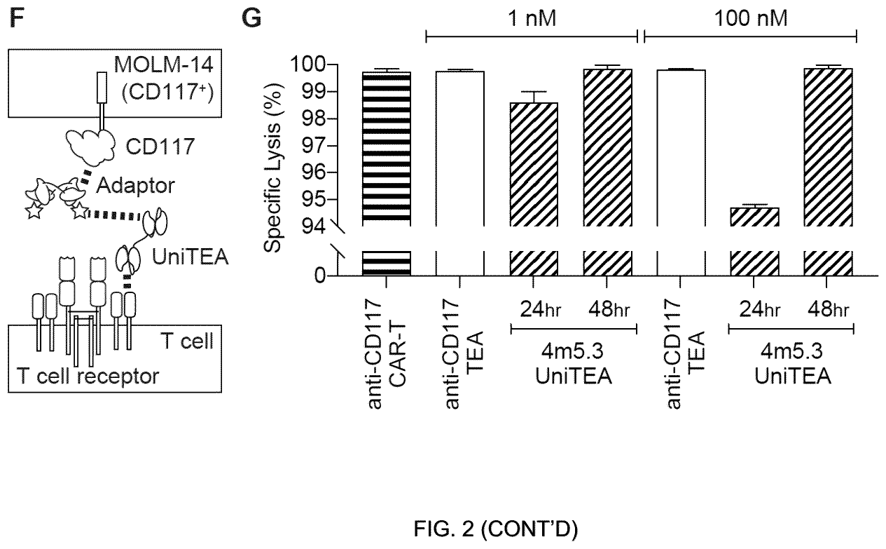

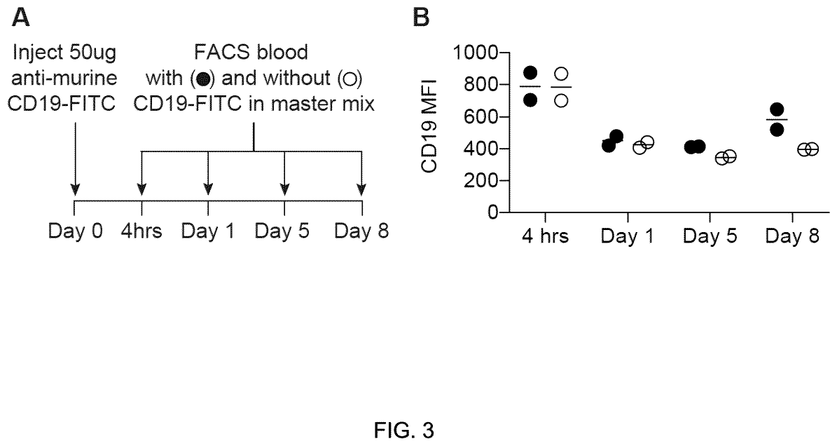

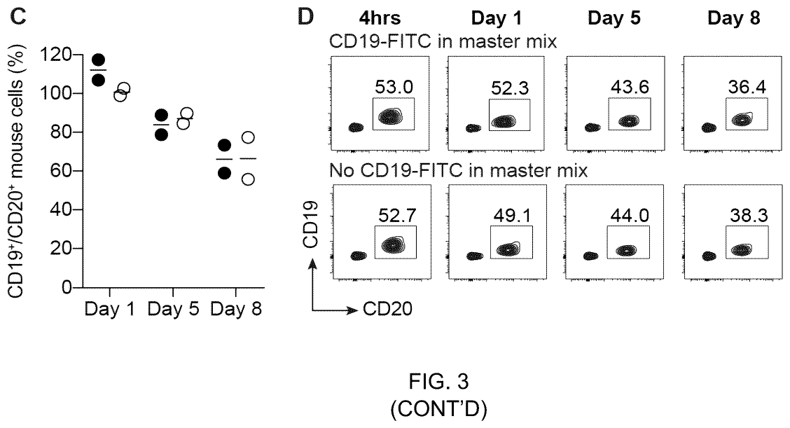

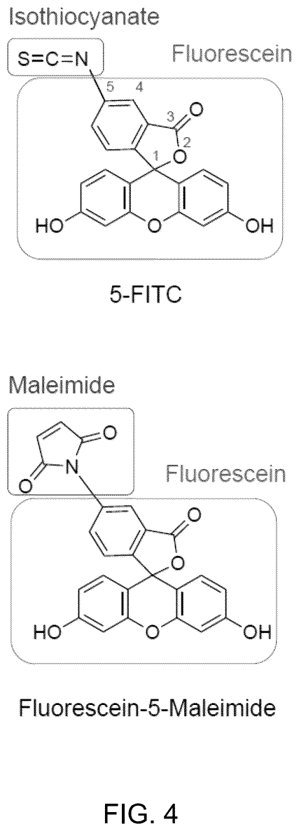

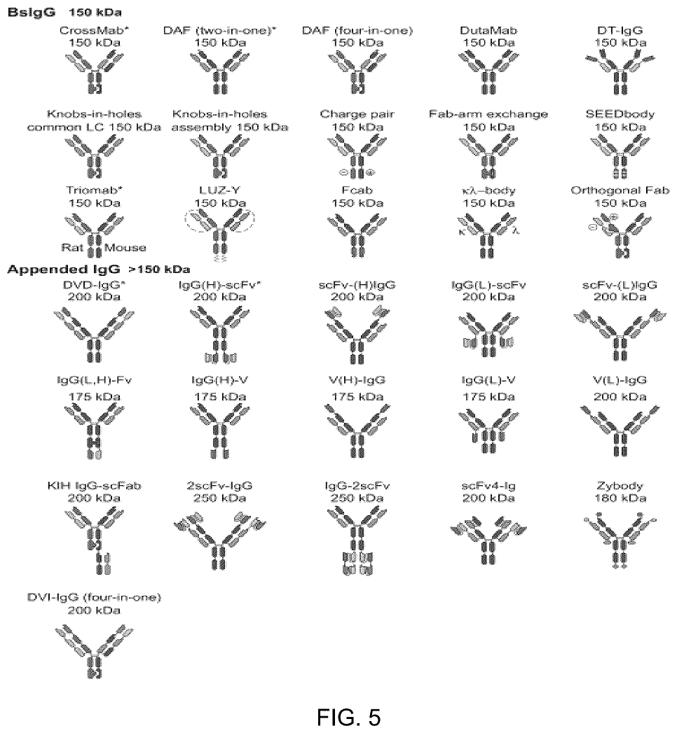

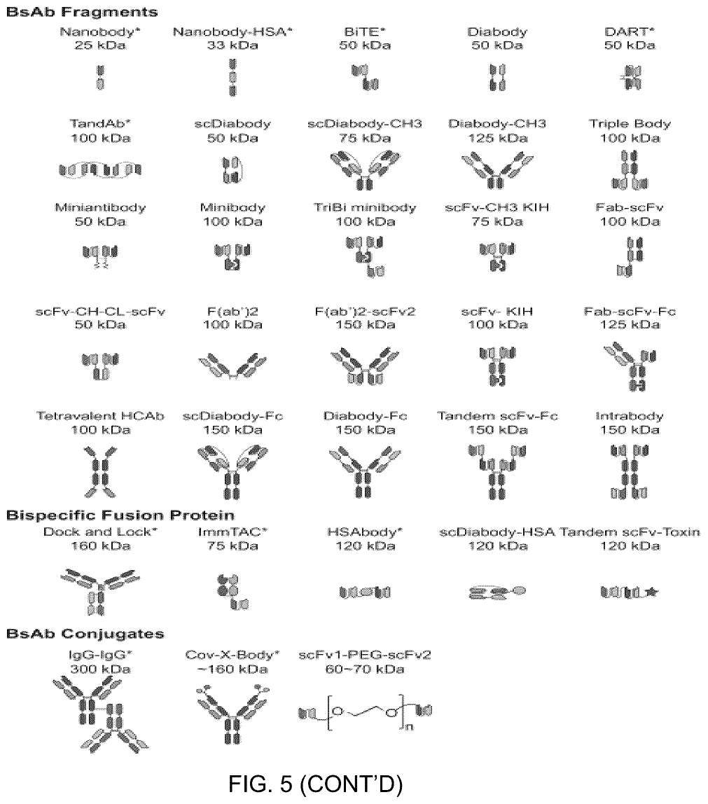

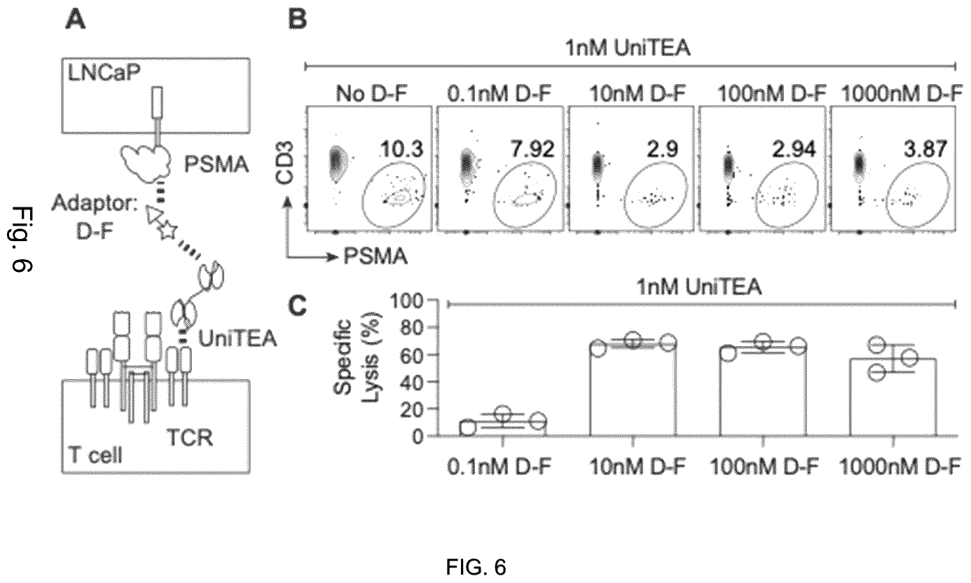

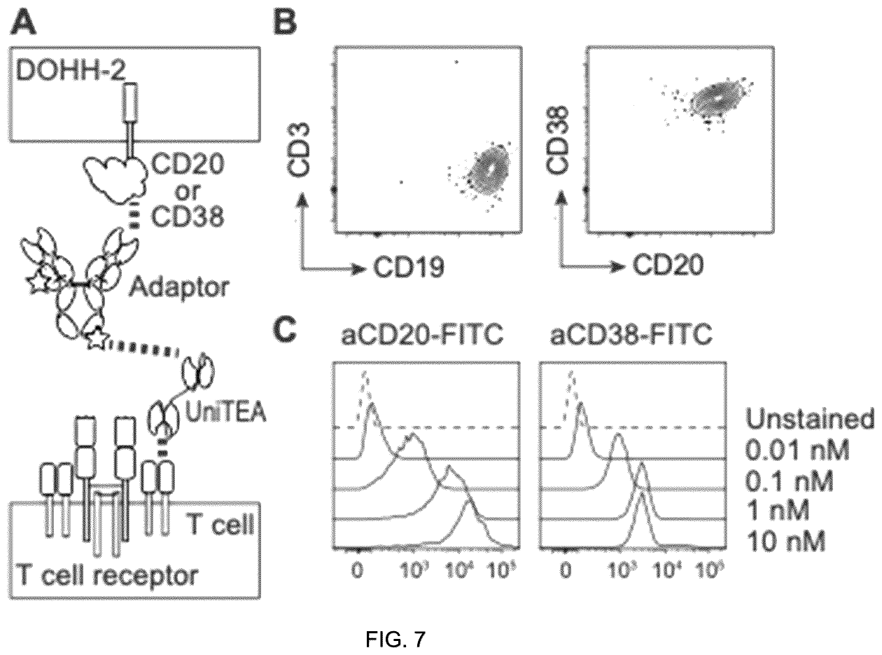

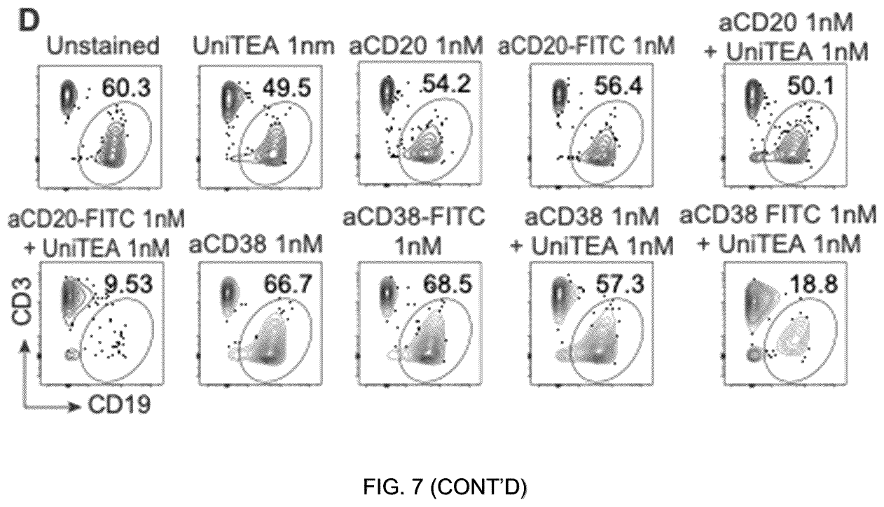

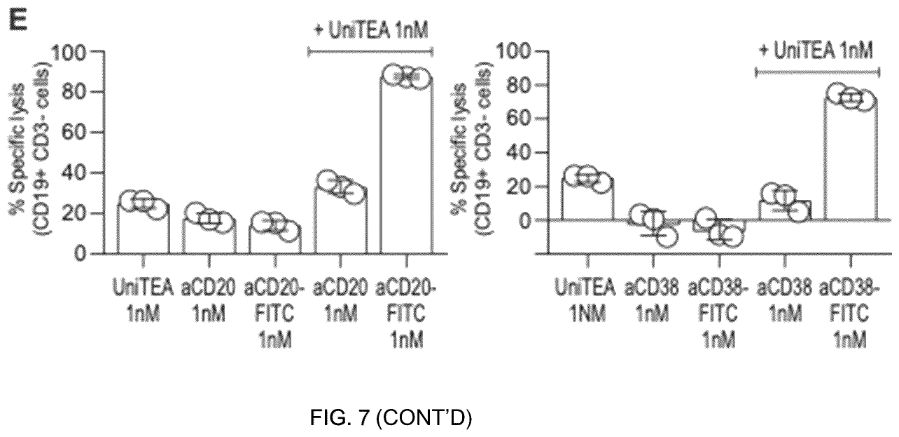

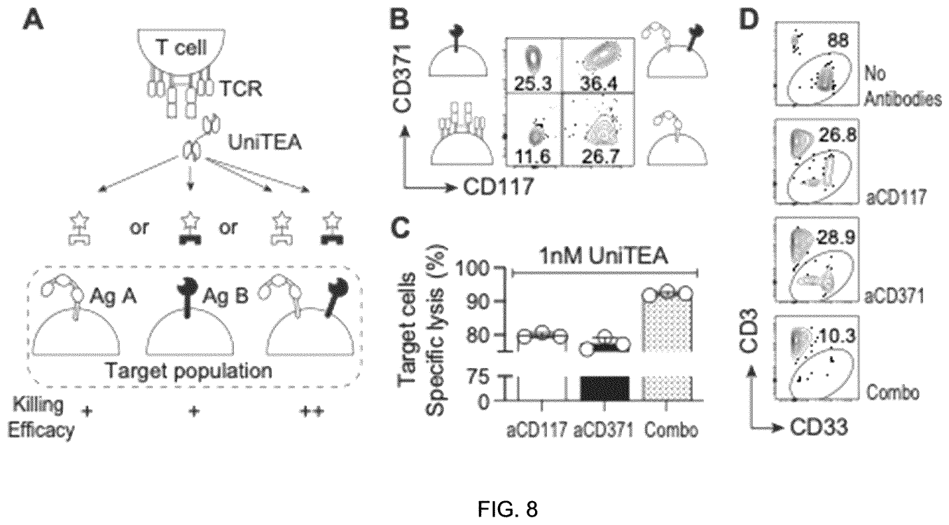

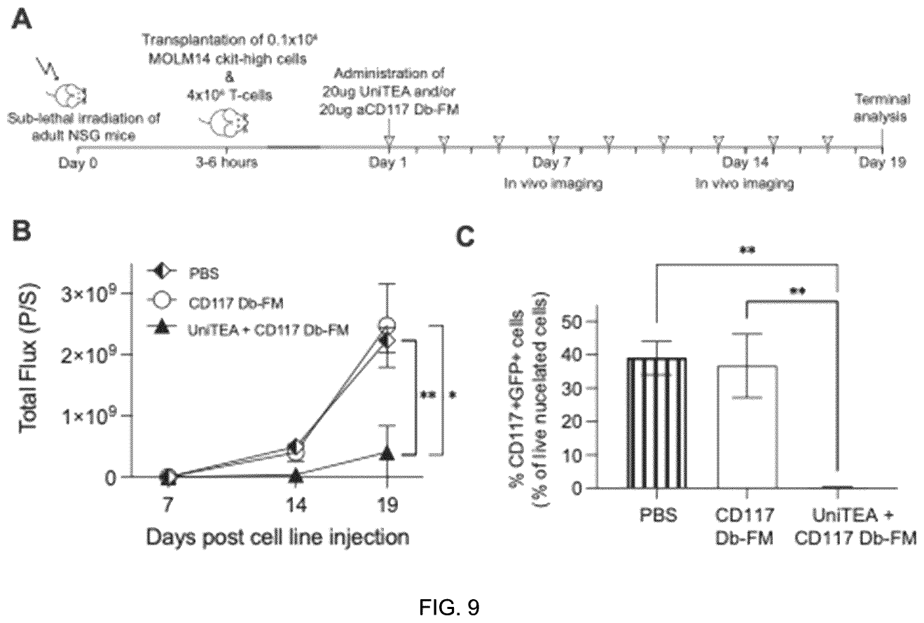

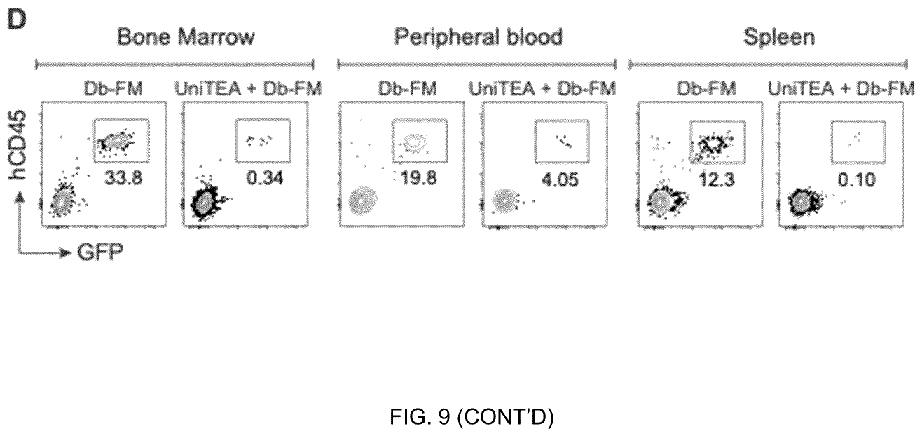

Aiming to re-direct, activate and induce T-cell mediated killing of potentially any target cell via a fluorescein-tagged target-binding molecules [FIG. 1A], we generated 3 versions of the UniTEs which all bind human CD3ε and fluorescein [FIG. 1B]. The 3 versions are designated as follows; 4m5.3/BlinCD3, E2/BlinCD3 and FluA/BlinCD3. Each UniTEA version differs in the fluorescein-binding moiety. 4m5.3 and E2 are antibody fragments in scFv format against fluorescein of mouse and human origin, respectively. FluA is a lipocalin fluorescein-binding molecule derived from Pieris brassicae.

UniTEs redirect T-cells to deplete CD19 and CD117-expressing leukemia cell lines in vitro Two UniTEs—4m5.3/BlinCD3 and FluA/BlinCD3—were tested functionally in vitro by determining their potential to redirect T cell killing against CD19⁺ and CD117⁺ human cell lines [FIG. 2]. The CD19⁺ NALM6 cell line was used to compare the T-cell mediated killing elicited by the 4m5.3/BlinCD3 and FluA/BlinCD3 UniTEA formats compared to the clinical-grade BiTE™ Blinatumomab (anti-CD19/BlinCD3) [FIG. 2A]. We confirm that T-cell activation and killing is elicited only in the presence of both the UniTEA and adaptor molecule since target cells and T-cells co-incubated with either UniTEA or bridge molecule alone led to minimal (˜5%) background lysis of target cells [FIG. 2B]. When using an effector to target ratio as high as 10:1, it is expected to see some spontaneous activity of the T cells against target cells in the absence and any T cell activating agent. After 24 hours, Blinatumomab resulted in over 50% and up to 77% T-cell mediated target cell lysis [FIG. 2C]. The addition of 4m5.3/BlinCD3 or FluA/BlinCD3 UniTEA could achieve similar target cell lysis of equimolar concentration of Blinatumomab when 10 nM of commercially available anti-CD19 IgG-FITC (Biolegend; clone HIB1) was used as bridging molecule [FIG. 2C]. The CD117⁺ MOLM14 cell line was used to compare the T-cell mediated killing elicited by the 4m5.3/BlinCD3 and FluA/BlinCD3 UniTEA formats compared to conventional CAR-T cells and BiTE™ directed against CD117 [FIG. 2D]. The CD117 targeting CAR T cell and BiTE™ could achieve >95% target cell lysis after 24 hours [FIG. 2E]. Similar target cell lysis was exhibited at the same time point when 100 nM of 4m5.3/BlinCD3 UniTEA were added in combination with 10 nM fluorescein-labeled anti-CD117 Db. The addition of 1 nM of 4m5.3/BlinCD3 UniTEA resulted in >95% target cell lysis after 48 hours [FIG. 2E].

### In Vivo Persistence of Fluoresceinated Antibodies

The stability in blood of the fluorescein over time is necessary for an efficient UniTE-fluoresceinated bridge complex formation and T-cell redirection. In order to assess the availability of structurally intact fluorescein on bridge molecules we administered 50ug of commercially available anti-murine CD19 (mCD19) IgG-FITC and analyzed the peripheral blood at different time points spanning 8 days [FIG. 3A]. At each time point analyzed, mCD19⁺ cells belonging to same mouse showed no difference in mean fluorescence intensity (MFI) when stained with or without additional mCD19-FITC [FIG. 3B]. However, the MFI of the mCD19⁺ population decreased after 24 hours but then remained stable until day 8 [FIG. 3B]. There was no difference in the proportion of mCD19⁺/mCD20⁺ B cells (expressed as percentage of mCD19⁺/mCD20⁺ cells compared to the baseline number after 4 hrs) stained with and without additional CD19-FITC labelling [FIG. 3C]. Since additional CD19-FITC labelling for flow cytometry did not increase the proportion of CD19⁺ cells or the MFI, all available CD19 epitopes were still occupied by the injected CD19-FITC antibody at day 0 and FITC remains stable in vivo over 5 days. Only at day 8, additional mCD19-FITC labelling resulted in an increase of the MFI of the CD19⁺ population indicating that at day 8 some of the injected mCD19-FITC antibody had been cleared from the body. Representative FACS plots showing the peripheral blood mCD19⁺/mCD20⁺ B cell population at indicated time points with and without additional mCD19-FITC labelling are reported in FIG. 3D. These populations were pre-gated on live single cells.

### REFERENCES

Myburgh, R. et al. Anti-human CD117 CAR T-cells efficiently eliminate healthy and malignant CD117-expressing hematopoietic cells. Leukemia (2020).
Horn, L. A. et al. CD3xPDL1 bi-specific T cell engager (BiTE) simultaneously activates T cells and NKT cells, kills PDL1+ tumor cells, and extends the survival of tumor-bearing humanized mice. Oncotarget 8, 57964 (2017).
Boder, E. T., Midelfort, K. S. & Wittrup, K. D. Directed evolution of antibody fragments with monovalent femtomolar antigen-binding affinity. Proc. Natl. Acad. Sci. 97, 10701-10705 (2000).
Vaughan, T. J. et al. Human Antibodies with Sub-nanomolar Affinities Isolated from a Large Non-immunized Phage Display Library. Nat. Biotechnol. 14, 309-314 (1996).
Vopel, S., Mühlbach, H. & Skerra, A. Rational engineering of a fluorescein-binding anticalin for improved ligand affinity. Biol. Chem. 386, 1097-1104 (2005).
Reshetnyak, A. V. et al. Structural basis for KIT receptor tyrosine kinase inhibition by antibodies targeting the D4 membrane-proximal region. Proc. Natl. Acad. Sci. U. S. A. 110, 17832-17837 (2013).
Casi, G., Huguenin-Dezot, N., Zuberbühler, K., Scheuermann, J. & Neri, D. Site-specific traceless coupling of potent cytotoxic drugs to recombinant antibodies for pharmacodelivery. J. Am. Chem. Soc. 134, 5887-5892 (2012).
Rajendra, Y., Kiseljak, D., Baldi, L., Hacker, D. L. & Wurm, F. M. A simple high-yielding process for transient gene expression in CHO cells. J. Biotechnol. 153, 22-26 (2011).
Plückthun and Skerra, Meth. Enzymol., 178:497-515 (1989) and in Day, E. D., Advanced Immunochemistry, Second Ed., Wiley-Liss, Inc., New York, N.Y. (1990)
Huston et al., Cell Biophysics, 22:189-224 (1993)
Pellegrino et al, Bioconjugate Chem. 2020, 31, 1775-1783
Eylenstein R, Weinfurtner D, Härtle S, Strohner R, Böttcher J, Augustin M, Ostendorp R, Steidl S. Molecular basis of in vitro affinity maturation and functional evolution of a neutralizing anti-human GM-CSF antibody. MAbs. 2016; 8(1):176-86
Harding F A, Stickler M M, Razo J, DuBridge R B. The immunogenicity of humanized and fully human antibodies: residual immunogenicity resides in the CDR regions. MAbs. 2010 May-June; 2(3):256-65.
Schanzer, J. M., et al., A novel glycoengineered bispecific antibody format for targeted inhibition of epidermal growth factor receptor (EGFR) and insulin-like growth factor receptor type I (IGF-1R) demonstrating unique molecular properties. J Biol Chem, 2014. 289(27): p. 18693-706.
Goebeler, M. E. and R. Bargou, Blinatumomab: a CD19/CD3 bispecific T cell engager (BiTE) with unique anti-tumor efficacy. Leuk Lymphoma, 2016. 57(5): p. 1021-32.
Neri, D., et al., High-affinity antigen binding by chelating recombinant antibodies (CRAbs). J Mol Biol, 1995. 246(3): p. 367-73.
Nagorsen, D., et al., Blinatumomab: a historical perspective. Pharmacol Ther, 2012. 136(3): p. 334-42
Cazzamalli S. et al, Chemically Defined Antibody- and Small Molecule-Drug Conjugates for in vivo Tumor Targeting Applications: A Comparative Analysis. J Am Chem Soc. 2018 Feb. 7; 140(5):1617-1621
Spiess C et al., Alternative molecular formats and therapeutic applications for bispecific antibodies. Mol Immunol. 2015 October; 67(2 Pt A):95-106.
Wang, A systematic approach for analysis and characterization of mispairing in bispecific antibodies with asymmetric architecture. MAbs. 2018 November-December; 10(8):1226-1235
Ceuppens J L et al, CD3 modulation inhibits pokeweed mitogen-induced T-cell help for immunoglobulin production. Cell Immunol. 1986 Oct. 1; 102(1):144-51.
Borst J et al., BMA031, a monoclonal antibody suited to identify the T-cell receptor alpha beta/CD3 complex on viable human T lymphocytes in normal and disease states. Hum Immunol. 1990 November; 29(3):175-88.
Li B et al., Construction and characterization of a humanized anti-human CD3 monoclonal antibody 12F6 with effective immunoregulation functions. Immunology. 2005 December; 116(4):487-98.

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| No | Sequence | Type |
|---|---|---|
| 1 | DIKLQQSGAELARPGASVKMSCKTSGYTFTRYTMHWVKQRPGQGLEWIGY | HCVD of anti CD3 |
| | INPSRGYTNYNQKFKDKATLTTDKSSSTAYMQLSSLTSEDSAVYYCARYY | BlinCD3 |
| | DDHYCLDYWGQGTTLTVSS | |
| 2 | YTFTRYTMH | HCDR1 of anti CD3 |
| | | BlinCD3 |
| 3 | WIGYINPSRGYTNY | HCDR2 of anti CD3 |
| | | BlinCD3 |
| 4 | RYYDDHYCLDY | HCDR3 of anti CD3 |
| | | BlinCD3 |
| 5 | GYTFTRY | alternative HCDR1 of anti |
| | | CD3 BlinCD3 |
| 6 | NPSRGY | alternative HCDR2 of anti |
| | | CD3 BlinCD3 |
| 7 | YYDDHYCLDY | alternative HCDR3 of anti |
| | | CD3 BlinCD3 |
| 8 | DIQLTQSPAIMSASPGEKVTMTCRASSSVSYMNWYQQKSGTSPKRWIYDT | LCVD of anti CD3 BlinCD3 |
| | SKVASGVPYRFSGSGSGTSYSLTISSMEAEDAATYYCQQWSSNPLTFGAG | |
| | TKLELK | |
| 9 | SSVSYMN | LCDR1 of anti CD3 |
| | | BlinCD3 |
| 10 | RWIYDTSKVAS | LCDR2 of anti CD3 |
| | | BlinCD3 |
| 11 | QQWSSNPL | LCDR3 of anti CD3 |
| | | BlinCD3 |
| 12 | RASSSVSYMN | alternative LCDR1 of anti |
| | | CD3 BlinCD3 |
| 13 | DTSKVAS | alternative LCDR2 of anti |
| | | CD3 BlinCD3 |
| 14 | QQWSSNPLT | alternative LCDR3 of anti |
| | | CD3 BlinCD3 |
| 15 | DIKLQQSGAELARPGASVKMSCKTSGYTFTRYTMHWVKQRPGQGLEWIGY | scFV sequence of anti |
| | INPSRGYTNYNQKFKDKATLTTDKSSSTAYMQLSSLTSEDSAVYYCARYY | CD3 (VH-Linker-VL) |
| | DDHYCLDYWGQGTTLTVSSVEGGSGGSGGSGGSGGVDDIQLTQSPAIMSA | BlinCD3 |
| | SPGEKVTMTCRASSSVSYMNWYQQKSGTSPKRWIYDTSKVASGVPYRFSG | |
| | SGSGTSYSLTISSMEAEDAATYYCQQWSSNPLTFGAGTKLELK | |
| 16 | QVQLQQSGAELARPGASVKMSCKASGYTFTRYTMHWVKQRPGQGLEWIGY | HCVD of anti CD3 OKT3 |
| | INPSRGYTNYNQKFKDKATLTTDKSSSTAYMQLSSLTSEDSAVYYCARYY | |
| | DDHYCLDYWGQGTTLTVSS | |
| 17 | YTFTRYTMH | HCDR1 of anti CD3 OKT3 |
| 18 | WIGYINPSRGYTNY | HCDR2 of anti CD3 OKT3 |
| 19 | RYYDDHYCLDY | HCDR3 of anti CD3 OKT3 |
| 20 | QIVLTQSPAIMSASPGEKVTMTCSASSSVSYMNWYQQKSGTSPKRWIYDT | LCVD of anti CD3 OKT3 |
| | SKLASGVPAHFRGSGSGTSYSLTISGMEAEDAATYYCQQWSSNPFTFGSG | |
| | TKLEINR | |
| 21 | SSVSYMN | LCDR1 of anti CD3 OKT3 |
| 22 | RWIYDTSKLAS | LCDR2 of anti CD3 OKT3 |
| 23 | QQWSSNPF | LCDR3 of anti CD3 OKT3 |
| 24 | QVQLQQSGAELARPGASVKMSCKASGYTFTRYTMHWVKQRPGQGLEWIGY | scFV sequence of anti |
| | INPSRGYTNYNQKFKDKATLTTDKSSSTAYMQLSSLTSEDSAVYYCARYY | CD3 (VH-Linker-VL) |
| | DDHYCLDYWGQGTTLTVSSVEGGSGGSGGSGGSGGVDQIVLTQSPAIMSA | |
| | SPGEKVTMTCSASSSVSYMNWYQQKSGTSPKRWIYDTSKLASGVPAHERG | |
| | SGSGTSYSLTISGMEAEDAATYYCQQWSSNPFTFGSGTKLEINR | |
| 25 | MGWSLILLELVAVATGVHS | Signal peptide |
| 26 | VEGGSGGSGGSGGSGGVD | Linker in BlinCD3 and Okt |
| | | 3 scFv |
| 27 | QVQLVESGGNLVQPGGSLRLSCAASGFTFGSFSMSWVRQAPGGGLEWVAG | HCVD of E2 (DP47) |
| | LSARSSLTHYADSVKGRFTISRDNAKNSVYLQMNSLRVEDTAVYYCARRS | |
| | YDSSGYWGHFYSYMDVWGQGTLVTVS | |
| 28 | FTFGSFSMS | HCDR1 of E2 (DP47) |
| 29 | WVAGLSARSSLTHY | HCDR2 of E2 (DP47) |
| 30 | RSYDSSGYWGHFYSYMDVW | HCDR3 of (DP47) |
| 31 | QSVLTQPSSVSAAPGQKVTISCSGSTSNIGNNYVSWYQQHPGKAPKLMIY | LCVD of E2 (DPL5/2) |
| | DVSKRPSGVPDRESGSKSGNSASLDISGLQSEDEADYYCAAWDDSLSEFL | |
| | FGTGTKLTVL | |
| 32 | TSNIGNNYVS | LCDR1 of E2 (DPL5/2) |
| 33 | LMIYDVSKRPS | LCDR2 of E2 (DPL5/2) |
| 34 | AAWDDSLSEFL | LCDR3 of E2 (DPL5/2) |
| 35 | QVQLVESGGNLVQPGGSLRLSCAASGFTFGSFSMSWVRQAPGGGLEWVAG | scFV sequence of E2 (VH- |
| | LSARSSLTHYADSVKGRFTISRDNAKNSVYLQMNSLRVEDTAVYYCARRS | Linker-VL) |
| | YDSSGYWGHFYSYMDVWGQGTLVTVS | |
| | GGGGSGGGGSGGGGS | |
| | QSVLTQPSSVSAAPGQKVTISCSGSTSNIGNNYVSWYQQHPGKAPKLMIY | |
| | DVSKRPSGVPDRESGSKSGNSASLDISGLQSEDEADYYCAAWDDSLSEFL | |
| | FGTGTKLTVL | |
| 36 | DVVMTQTPLSLPVSLGDQASISCRSSQSLVHSNGNTYLRWYLQKPGQSPK | HCVD of 4m5.3 |
| | VLIYKVSNRVSGVPDRESGSGSGTDFTLKINRVEAEDLGVYFCSQSTHVP | |
| | WTFGGGTKLEIKSS | |
| 37 | QSLVHSNGNTYLR | HCDR1 of 4m5.3 |
| 38 | LIYKVSNRVS | HCDR2 of 4m5.3 |
| 39 | SQSTHVPW | HCDR3 of 4m5.3 |
| 40 | VKLDETGGGLVQPGGAMKLSCVTSGFTFGHYWMNWVRQSPEKGLEWVAQF | LCVD of 4m5.3 |
| | RNKPYNYETYYSDSVKGRFTISRDDSKSSVYLQMNNLRVEDTGIYYCTGA | |
| | SYGMEYLGQGTSVTVS | |
| 41 | FTFGHYWMN | LCDR1 of 4m5.3 |
| 42 | WVAQFRNKPYNYETYY | LCDR2 of 4m5.3 |
| 43 | GASYGMEYL | LCDR3 of 4m5.3 |
| 44 | DVVMTQTPLSLPVSLGDQASISCRSSQSLVHSNGNTYLRWYLQKPGQSPK | scFV sequence of 4m5.3 |
| | VLIYKVSNRVSGVPDRFSGSGSGTDFTLKINRVEAEDLGVYFCSQSTHVP | (VH-Linker-VL) |
| | WTFGGGTKLEIKSS | |
| | ADDAKKDAAKKDDAKKDDAKKDGG | |
| | VKLDETGGGLVQPGGAMKLSCVTSGFTFGHYWMNWVRQSPEKGLEWVAQF | |
| | RNKPYNYETYYSDSVKGRFTISRDDSKSSVYLQMNNLRVEDTGIYYCTGA | |
| | SYGMEYLGQGTSVTVS | |
| 45 | GGGGSGGGGSGGGGS | linker in E2 |
| 46 | ADDAKKDAAKKDDAKKDDAKKDGG | Linker in 4m5.3 scFv |
| 47 | DVYHDGACPEVKPVDNEDWSQYHGKWWEVAKYPSPNGKYGKCGWIEYTPE | Anticalin FluA |
| | GKSVKVSRYDVIHGKEYFMEGTAYPVGDSKIGKIYHSRTVGGYTKKTVEN | (R95K/A45I/S114R |
| | VLSTDNKNYIIGYRCRYDEDKKGHWDHVWVLSRSMVLTGEAKTAVENYLI | variant) |
| | GSPVVDSQKLVYSDESEAACKVNNSNWSHPQFEK | |
| 48 | QVQLVESGGNLVQPGGSLRLSCAASGFTFGSFSMSWVRQAPGGGLEWVAG | Human VH (DP47)- |
| | LSARSSLTHYADSVKGRFTISRDNAKNSVYLQMNSLRVEDTAVYYCARRS | (GGGGS)3 linker- |
| | YDSSGYWGHFYSYMDVWGQGTLVTVS | Human VL (DPL5/2)- |
| | GGGGSGGGGSGGGGS | (GGGGS) linker- |
| | QSVLTQPSSVSAAPGQKVTISCSGSTSNIGNNYVSWYQQHPGKAPKLMIY | OKT3opt VH-linker- |
| | DVSKRPSGVPDRESGSKSGNSASLDISGLQSEDEADYYCAAWDDSLSEFL | OKT3opt VL 6xHis tag |
| | FGTGTKLTVL | |
| | GGGGS | |
| | DIKLQQSGAELARPGASVKMSCKTSGYTFTRYTMHWVKQRPGQGLEWIGY | |
| | INPSRGYTNYNQKFKDKATLTTDKSSSTAYMQLSSLTSEDSAVYYCARYY | |
| | DDHYCLDYWGQGTTLTVSS | |
| | VEGGSGGSGGSGGSGGVD | |
| | DIQLTQSPAIMSASPGEKVTMTCRASSSVSYMNWYQQKSGTSPKRWIYDT | |
| | SKVASGVPYRFSGSGSGTSYSLTISSMEAEDAATYYCQQWSSNPLTFGAG | |
| | TKLELK | |
| | HHHHHH | |
| 49 | DVVMTQTPLSLPVSLGDQASISCRSSQSLVHSNGNTYLRWYLQKPGQSPK | Murine VL (4m5.3)- |
| | VLIYKVSNRVSGVPDRESGSGSGTDFTLKINRVEAEDLGVYFCSQSTHVP | original linker-Murine |
| | WTFGGGTKLEIKSS | VH (4m5.3)-(GGGGS) |
| | ADDAKKDAAKKDDAKKDDAKKDGG | linker-OKT3opt VH- |
| | VKLDETGGGLVQPGGAMKLSCVTSGFTFGHYWMNWVRQSPEKGLEWVAQF | linker-OKT3opt VL |
| | RNKPYNYETYYSDSVKGRFTISRDDSKSSVYLQMNNLRVEDTGIYYCTGA | 6xHis tag |
| | SYGMEYLGQGTSVTVS | |
| | GGGGS | |
| | DIKLQQSGAELARPGASVKMSCKTSGYTFTRYTMHWVKQRPGQGLEWIGY | |
| | INPSRGYTNYNQKFKDKATLTTDKSSSTAYMQLSSLTSEDSAVYYCARYY | |
| | DDHYCLDYWGQGTTLTVSS | |
| | VEGGSGGSGGSGGSGGVD | |
| | DIQLTQSPAIMSASPGEKVTMTCRASSSVSYMNWYQQKSGTSPKRWIYDT | |
| | SKVASGVPYRFSGSGSGTSYSLTISSMEAEDAATYYCQQWSSNPLTFGAG | |
| | TKLELK | |
| | HHHHHH | |
| 50 | DVYHDGACPEVKPVDNFDWSQYHGKWWEVAKYPSPNGKYGKCGWIEYTPE | FluA-(GGGGS) linker- |
| | GKSVKVSRYDVIHGKEYFMEGTAYPVGDSKIGKIYHSRTVGGYTKKTVEN | OKT3opt VH-linker- |
| | VLSTDNKNYIIGYRCRYDEDKKGHWDHVWVLSRSMVLTGEAKTAVENYLI | OKT3opt VL 6xHis tag |
| | GSPVVDSQKLVYSDESEAACKVNNSNWSHPQFEK | |
| | GGGGS | |
| | DIKLQQSGAELARPGASVKMSCKTSGYTFTRYTMHWVKQRPGQGLEWIGY | |
| | INPSRGYTNYNQKFKDKATLTTDKSSSTAYMQLSSLTSEDSAVYYCARYY | |
| | DDHYCLDYWGQGTTLTVSS | |
| | VEGGSGGSGGSGGSGGVD | |
| | DIQLTQSPAIMSASPGEKVTMTCRASSSVSYMNWYQQKSGTSPKRWIYDT | |
| | SKVASGVPYRFSGSGSGTSYSLTISSMEAEDAATYYCQQWSSNPLTFGAG | |
| | TKLELK | |
| | HHHHHH | |
| 51 | DIKLQQSGAELARPGASVKMSCKTSGYTFTRYTMHWVKQRPGQGLEWIGY | OKT3opt VH-linker- |
| | INPSRGYTNYNQKFKDKATLTTDKSSSTAYMQLSSLTSEDSAVYYCARYY | OKT3opt VL-Ala₃-Myc |
| | DDHYCLDYWGQGTTLTVSS | tag-6xHis tag- |
| | VEGGSGGSGGSGGSGGVD | Cysteine |
| | DIQLTQSPAIMSASPGEKVTMTCRASSSVSYMNWYQQKSGTSPKRWIYDT | |
| | SKVASGVPYRFSGSGSGTSYSLTISSMEAEDAATYYCQQWSSNPLTFGAG | |
| | TKLELK | |
| | AAA | |
| | EQKLISEEDLGS | |
| | HHHHHH | |
| | C | |
| 52 | SEVQLVESGGGLVQPGGSLRLSCAASGENISVYMMHWVRQAPGKGLEWVA | HCVD of anti CD117 79D |
| | SIYPYSGYTYYADSVKGRFTISADTSKNTAYLQMNSLRAEDTAVYYCARY | |
| | VYHALDYWGQGTLVTVSS | |
| 53 | FNISVYMMH | HCDR1 of anti CD117 |
| | | 79D |
| 54 | WVASIYPYSGYTYY | HCDR2 of anti CD117 |
| | | 79D |
| 55 | RYVYHALDY | HCDR3 of anti CD117 |
| | | 79D |
| 56 | GENISVY | alternative HCDR1 of anti |
| | | CD117 79D |
| 57 | YPYSGY | alternative HCDR2 of anti |
| | | CD117 79D |
| 58 | YVYHALDY | alternative HCDR3 of anti |
| | | CD117 79D |
| 59 | DIQMTQSPSSLSASVGDRVTITCRASQRGLRNVAVAWYQQKPGKAPKLLI | LCVD of anti CD117 79D |
| | YSASSLYSGVPSRESGSRSGTDFTLTISSLQPEDFATYYCQQWAVHSLIT | |
| | FGQGTKVEIKR | |
| 60 | QRGLRNVAVA | LCDR1 of anti CD117 79D |
| 61 | LLIYSASSLYS | LCDR2 of anti CD117 79D |
| 62 | QQWAVHSLI | LCDR3 of anti CD117 79D |
| 63 | RASQRGLRNVAVA | alternative LCDR1 of anti |
| | | CD117 79D |
| 64 | SASSLYS | alternative LCDR2 of anti |
| | | CD117 79D |
| 65 | QQWAVHSLIT | alternative LCDR3 of anti |
| | | CD117 79D |
| 66 | DIQMTQSPSSLSASVGDRVTITCRASQRGLRNVAVAWYQQKPGKAPKLLI | scFV sequence of anti |
| | YSASSLYSGVPSRESGSRSGTDFTLTISSLQPEDFATYYCQQWAVHSLIT | CD117 (VL-Linker-VH) |
| | FGQGTKVEIKRGGGGSGGGGSGGGGSEVQLVESGGGLVQPGGSLRLSCAA | |
| | SGENISVYMMHWVRQAPGKGLEWVASIYPYSGYTYYADSVKGRFTISADT | |
| | SKNTAYLQMNSLRAEDTAVYYCARYVYHALDYWGQGTLVTVSS | |
| 67 | GGGGS | linker |
| 68 | EVQLLESGGGLVQPGGSLRLSCAASGFTFSSYAMSWVRQAPGKGLEWVSA | HCVD of anti CAIX |
| | IDGSGGSTYYADSVKGRFTISRDNSKNTLYLQMNSLRAEDTAVYYCVKGP | (XE114) |
| | PVFDYWGQGTLVTVSS | |
| 69 | FTFSSYAMS | HCDR1 of anti CAIX |
| | | XE114 |
| 70 | WVSAIDGSGGSTYYA | HCDR2 of anti CAIX |
| | | XE114 |
| 71 | VKGPPVEDY | HCDR3 of anti CAIX |
| | | XE114 |
| 72 | SSELTQDPAVSVALGQTVRITCQGDSLRSYYASWYQQKPGQAPVLVIYGK | HCVD of anti CAIX |
| | NNRPSGIPDRESGSSSGNTASLTITGAQAEDEADYYCQSSKWSWDPVVFG | (XE114) |
| | GGTKLTVL | |
| 73 | SLRSYYAS | LCDR1 of anti CAIX |
| | | XE114 |
| 74 | LVIYGKNNRPS | LCDR2 of anti CAIX |
| | | XE114 |
| 75 | QSSKWSWDPV | LCDR3 of anti CAIX |
| | | XE114 |

1. A bispecific antibody comprising at least a first binding domain and a second binding domain, wherein said first binding domain binds to an organic fluorophore and wherein said second binding domain binds to CD3.

2. The bispecific antibody according to claim 1, wherein said first binding domain binds to Fluorescein or Fluorescein when bound to a protein (“conjugated Fluorescein”).

3. The bispecific antibody according to claim 2, wherein the first binding domain which binds to Fluorescein or conjugated Fluorescein
a) comprises a set of heavy chain/light chain complementarity determining regions (CDR) comprised in the heavy chain/light variable region sequence pair of one of the anti Fluorescein antibodies E2 or 4m5.3, as set forth in SEQ ID NOs 27 and 31, or SEQ ID NOs 36 and 40, respectively;
b) comprises the set of heavy chain/light chain complementarity determining regions (CDR) comprising the following sequences;
HC CDR1 (SEQ ID NO 28)
HC CDR2 (SEQ ID NO 29)
HC CDR3 (SEQ ID NO 30)
LC CDR1 (SEQ ID NO 32)
LC CDR2 (SEQ ID NO 33), and
LC CDR3 (SEQ ID NO 34);
c) comprises the set of heavy chain/light chain complementarity determining regions (CDR) comprising the following sequences
HC CDR1 (SEQ ID NO 37)
HC CDR2 (SEQ ID NO 38)
HC CDR3 (SEQ ID NO 39)
LC CDR1 (SEQ ID NO 41)
LC CDR2 (SEQ ID NO 42), and
LC CDR3 (SEQ ID NO 43);
d) comprises the heavy chain/light chain complementarity determining regions (CDR) of b) or c), with the proviso that at least one of the CDRs has up to 3 amino acid substitutions relative to the respective SEQ ID NO 28-30, 32-34, 37-39 or 41-43, and/or
e) comprises the heavy chain/light chain complementarity determining regions (CDR) of b), with the proviso that at least one of the CDRs has a sequence identity of ≥66% to the respective SEQ ID NO 28-30, 32-34, 37-39 or 41-43,
wherein the CDRs are embedded in a suitable protein framework so as to be capable to bind to Fluorescein or conjugated Fluorescein.

4. The bispecific antibody according to claim 2, wherein the first binding domain which binds to Fluorescein or conjugated Fluorescein comprises
a) the variable domains of one antibody selected from the group consisting of E2 or 4m5.3;
b) the heavy chain/light chain variable domains (VD)
HC VD (SEQ ID NO 27 or 36), and
LC VD (SEQ ID NO 31 or 40);
c) the heavy chain/light chain variable domains (VD) of b), with the proviso that
the HCVD has a sequence identity of ≥80% to the respective SEQ ID NO 27 or 36, and/or
d) the LCVD has a sequence identity of ≥80% to the respective SEQ ID NO 31 or 40 the heavy chain/light chain variable domains (VD) of b), with the proviso that at least one of the HCVD or LCVD has up to 10 amino acid substitutions relative to the respective SEQ ID NOs,
wherein said bispecific antibody is still capable to bind to Fluorescein or conjugated Fluorescein.

5. The bispecific antibody according to claim 1, wherein the second binding domain which binds to CD3
a) comprises a set of heavy chain/light chain complementarity determining regions (CDR) comprised in the heavy chain/light variable region sequence pair of one of the anti CD3 antibodies BlinCD3 and OKT3, as set forth in SEQ ID NOs 1 and 8, or SEQ ID NOs 16 and 20, respectively;
b) comprises the set of heavy chain/light chain complementarity determining regions (CDR) comprising the following sequences
HC CDR1 (SEQ ID NO 2 or 5)
HC CDR2 (SEQ ID NO 3 or 6)
HC CDR3 (SEQ ID NO 4 or 7)
LC CDR1 (SEQ ID NO 9 or 12)
LC CDR2 (SEQ ID NO 10 or 13), and
LC CDR3 (SEQ ID NO 11 or 14);
c) comprises the set of heavy chain/light chain complementarity determining regions (CDR) comprising the following sequences
HC CDR1 (SEQ ID NO 17)
HC CDR2 (SEQ ID NO 18)
HC CDR3 (SEQ ID NO 19)
LC CDR1 (SEQ ID NO 21)
LC CDR2 (SEQ ID NO 22), and
LC CDR3 (SEQ ID NO 23);
d) comprises the heavy chain/light chain complementarity determining regions (CDR) of b) or c), with the proviso that at least one of the CDRs has up to 3 amino acid substitutions relative to the respective SEQ ID NO 2-4, 9-11, 17-19 or 21-23, and/or
e) comprises the heavy chain/light chain complementarity determining regions (CDR) of b), with the proviso that at least one of the CDRs has a sequence identity of ≥66% to the respective SEQ ID NO 2-4, 9-11, 17-19 or 21-23,
wherein the CDRs are embedded in a suitable protein framework so as to be capable to bind to CD3.

6. The bispecific antibody according to claim 1, wherein the second binding domain which binds to CD3 comprises
a) the variable domains of one antibody selected from the group consisting of OKT3 and BlinCD3;
b) the heavy chain/light chain variable domains (VD)
HC VD (SEQ ID NO 1 or 16), and
LC VD (SEQ ID NO 8 or 20);
c) the heavy chain/light chain variable domains (VD) of b), with the proviso that the HCVD has a sequence identity of ≥80% to the respective SEQ ID NO 1 or 16, and/or
d) the LCVD has a sequence identity of ≥80% to the respective SEQ ID NO 8 or 20 the heavy chain/light chain variable domains (VD) of b), with the proviso that at least one of the HCVD or LCVD has up to 10 amino acid substitutions relative to the respective SEQ ID NOs,
wherein said bispecific antibody is still capable to bind to CD3.

7. The bispecific antibody according to claim 1, wherein the second binding domain which binds to CD3
a) comprises a scFv-like sequence as set forth in any one of SEQ ID NOs 15 or 24, or
b) comprises an amino acid sequence that has a sequence identity of ≥80% to any one of SEQ ID NOs 15 or 24.

8. The bispecific antibody according to claim 2, wherein the first binding domain which binds to Fluorescein or conjugated Fluorescein
a) comprises a scFv-like sequence as set forth in any one of SEQ ID NOs 35 or 44, or
b) comprises an amino acid sequence that has a sequence identity of ≥80% to any one of SEQ ID NOs 35 or 44.

9. The bispecific antibody according to claim 1 which
a) comprises at least one of the sequences selected from SEQ ID NOs 48-50, or
b) comprises an amino acid sequence that has a sequence identity of ≥80% to SEQ ID NOs 48-50,
with optionally a C terminal cysteine residue and/or a His tag removed or in place.

10. A combination comprising
a) the bispecific antibody according to claim 1 and
b) a labelled binding agent that binds specifically to a target antigen,
wherein the labelled binding agent is labelled with an organic fluorophore that is specifically detected and/or bound by the first binding domain of the bispecific antibody.

11. The combination according to claim 10, wherein the organic fluorophore in the labelled binding agent is fluorescein.

12. The combination according to claim 10, wherein the labelled binding agent comprises an antibody, or a target binding fragment or derivative thereof.

13. A pharmaceutical composition comprising the combination according to claim 10 and optionally one or more pharmaceutically acceptable excipients.

14. A method of treating a human or animal, said method comprising the combination or pharmaceutical composition according to claim 10, wherein the human or animal is
being diagnosed for,
suffering from or
being at risk of
developing a neoplastic disease, or for the prevention of such condition.

15. The combination for use according to claim 10, wherein a) and b) are administered
consecutively, with a) before b) or b) before a), or
concomitantly.


SEQUENCE LISTING (75 sequences)

SEQ ID NO: 1 - protein, 119 aa, artificial sequence
  antibody sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 2 - protein, 9 aa, artificial sequence
  antibody sequence
YTFTRYTMH

SEQ ID NO: 3 - protein, 14 aa, artificial sequence
  antibody sequence
WIGYINPSRGYTNY

SEQ ID NO: 4 - protein, 11 aa, artificial sequence
  antibody sequence
RYYDDHYCLDY

SEQ ID NO: 5 - protein, 7 aa, artificial sequence
  antibody sequence
GYTFTRY

SEQ ID NO: 6 - protein, 6 aa, artificial sequence
  antibody sequence
NPSRGY

SEQ ID NO: 7 - protein, 10 aa, artificial sequence
  antibody sequence
YYDDHYCLDY

SEQ ID NO: 8 - protein, 106 aa, artificial sequence
  antibody sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 9 - protein, 7 aa, artificial sequence
  antibody sequence
SSVSYMN

SEQ ID NO: 10 - protein, 11 aa, artificial sequence
  antibody sequence
RWIYDTSKVAS

SEQ ID NO: 11 - protein, 8 aa, artificial sequence
  antibody sequence
QQWSSNPL

SEQ ID NO: 12 - protein, 10 aa, artificial sequence
  antibody sequence
RASSSVSYMN

SEQ ID NO: 13 - protein, 7 aa, artificial sequence
  antibody sequence
DTSKVAS

SEQ ID NO: 14 - protein, 9 aa, artificial sequence
  antibody sequence
QQWSSNPLT

SEQ ID NO: 15 - protein, 243 aa, artificial sequence
  antibody sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 16 - protein, 119 aa, artificial sequence [sequence omitted]

SEQ ID NO: 17 - protein, 9 aa, artificial sequence
  antibody sequence
YTFTRYTMH

SEQ ID NO: 18 - protein, 14 aa, artificial sequence
  antibody sequence
WIGYINPSRGYTNY

SEQ ID NO: 19 - protein, 11 aa, artificial sequence
  antibody sequence
RYYDDHYCLDY

SEQ ID NO: 20 - protein, 107 aa, artificial sequence
  antibody sequence
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 21 - protein, 7 aa, artificial sequence
  antibody sequence
SSVSYMN

SEQ ID NO: 22 - protein, 11 aa, artificial sequence
  antibody sequence
RWIYDTSKLAS

SEQ ID NO: 23 - protein, 8 aa, artificial sequence
  antibody sequence
QQWSSNPF

SEQ ID NO: 24 - protein, 244 aa, artificial sequence
  antibody sequence
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 25 - protein, 19 aa, artificial sequence
  antibody sequence
MGWSLILLFLVAVATGVHS

SEQ ID NO: 26 - protein, 18 aa, artificial sequence
  antibody sequence
VEGGSGGSGGSGGSGGVD

SEQ ID NO: 27 - protein, 126 aa, artificial sequence [sequence omitted]

SEQ ID NO: 28 - protein, 9 aa, artificial sequence
  antibody sequence
FTFGSFSMS

SEQ ID NO: 29 - protein, 14 aa, artificial sequence
  antibody sequence
WVAGLSARSSLTHY

SEQ ID NO: 30 - protein, 19 aa, artificial sequence
  antibody sequence
RSYDSSGYWGHFYSYMDVW

SEQ ID NO: 31 - protein, 110 aa, artificial sequence
  antibody sequence
QSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 32 - protein, 10 aa, artificial sequence
  antibody sequence
TSNIGNNYVS

SEQ ID NO: 33 - protein, 11 aa, artificial sequence
  antibody sequence
LMIYDVSKRPS

SEQ ID NO: 34 - protein, 11 aa, artificial sequence
  antibody sequence
AAWDDSLSEFL

SEQ ID NO: 35 - protein, 251 aa, artificial sequence
  antibody sequence
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGGGGSGGGGSGGGGSQSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 36 - protein, 114 aa, artificial sequence
  antibody sequence
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSS

SEQ ID NO: 37 - protein, 13 aa, artificial sequence
  antibody sequence
QSLVHSNGNTYLR

SEQ ID NO: 38 - protein, 10 aa, artificial sequence
  antibody sequence
LIYKVSNRVS

SEQ ID NO: 39 - protein, 8 aa, artificial sequence
  antibody sequence
SQSTHVPW

SEQ ID NO: 40 - protein, 116 aa, artificial sequence
  antibody sequence
VKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 41 - protein, 9 aa, artificial sequence
  antibody sequence
FTFGHYWMN

SEQ ID NO: 42 - protein, 16 aa, artificial sequence
  antibody sequence
WVAQFRNKPYNYETYY

SEQ ID NO: 43 - protein, 9 aa, artificial sequence
  antibody sequence
GASYGMEYL

SEQ ID NO: 44 - protein, 254 aa, artificial sequence
  antibody sequence
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 45 - protein, 15 aa, artificial sequence
  antibody sequence
GGGGSGGGGSGGGGS

SEQ ID NO: 46 - protein, 24 aa, artificial sequence
  antibody sequence
ADDAKKDAAKKDDAKKDDAKKDGG

SEQ ID NO: 47 - protein, 184 aa, artificial sequence
  antibody sequence
DVYHDGACPEVKPVDNFDWSQYHGKWWEVAKYPSPNGKYGKCGWIEYTPEGKSVKVSRYDVIHGKEYFMEGTAYPVGDSKIGKIYHSRTVGGYTKKTVFNVLSTDNKNYIIGYRCRYDEDKKGHWDHVWVLSRSMVLTGEAKTAVENYLIGSPVVDSQKLVYSDFSEAACKVNNSNWSHPQFEK

SEQ ID NO: 48 - protein, 505 aa, artificial sequence
  antibody sequence
QVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSGGGGSGGGGSGGGGSQSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 49 - protein, 508 aa, artificial sequence
  antibody sequence
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 50 - protein, 438 aa, artificial sequence
  antibody sequence
DVYHDGACPEVKPVDNFDWSQYHGKWWEVAKYPSPNGKYGKCGWIEYTPEGKSVKVSRYDVIHGKEYFMEGTAYPVGDSKIGKIYHSRTVGGYTKKTVFNVLSTDNKNYIIGYRCRYDEDKKGHWDHVWVLSRSMVLTGEAKTAVENYLIGSPVVDSQKLVYSDFSEAACKVNNSNWSHPQFEKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 51 - protein, 265 aa, artificial sequence
  antibody sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAAEQKLISEEDLGSHHHHHHC

SEQ ID NO: 52 - protein, 118 aa, artificial sequence
  antibody sequence
SEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSS

SEQ ID NO: 53 - protein, 9 aa, artificial sequence
  antibody sequence
FNISVYMMH

SEQ ID NO: 54 - protein, 14 aa, artificial sequence
  antibody sequence
WVASIYPYSGYTYY

SEQ ID NO: 55 - protein, 9 aa, artificial sequence
  antibody sequence
RYVYHALDY

SEQ ID NO: 56 - protein, 7 aa, artificial sequence
  antibody sequence
GFNISVY

SEQ ID NO: 57 - protein, 6 aa, artificial sequence
  antibody sequence
YPYSGY

SEQ ID NO: 58 - protein, 8 aa, artificial sequence
  antibody sequence
YVYHALDY

SEQ ID NO: 59 - protein, 111 aa, artificial sequence
  antibody sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKR

SEQ ID NO: 60 - protein, 10 aa, artificial sequence
  antibody sequence
QRGLRNVAVA

SEQ ID NO: 61 - protein, 11 aa, artificial sequence
  antibody sequence
LLIYSASSLYS

SEQ ID NO: 62 - protein, 9 aa, artificial sequence
  antibody sequence
QQWAVHSLI

SEQ ID NO: 63 - protein, 13 aa, artificial sequence
  antibody sequence
RASQRGLRNVAVA

SEQ ID NO: 64 - protein, 7 aa, artificial sequence
  antibody sequence
SASSLYS

SEQ ID NO: 65 - protein, 10 aa, artificial sequence
  antibody sequence
QQWAVHSLIT

SEQ ID NO: 66 - protein, 243 aa, artificial sequence
  antibody sequence
DIQMTQSPSSLSASVGDRVTITCRASQRGLRNVAVAWYQQKPGKAPKLLIYSASSLYSGVPSRFSGSRSGTDFTLTISSLQPEDFATYYCQQWAVHSLITFGQGTKVEIKRGGGGSGGGGSGGGGSEVQLVESGGGLVQPGGSLRLSCAASGFNISVYMMHWVRQAPGKGLEWVASIYPYSGYTYYADSVKGRFTISADTSKNTAYLQMNSLRAEDTAVYYCARYVYHALDYWGQGTLVTVSS

SEQ ID NO: 67 - protein, 5 aa, artificial sequence
  antibody sequence
GGGGS

SEQ ID NO: 68 - protein, 116 aa, artificial sequence
  antibody sequence
EVQLLESGGGLVQPGGSLRLSCAASGFTFSSYAMSWVRQAPGKGLEWVSAIDGSGGSTYYADSVKGRFTISRDNSKNTLYLQMNSLRAEDTAVYYCVKGPPVFDYWGQGTLVTVSS

SEQ ID NO: 69 - protein, 9 aa, artificial sequence
  antibody sequence
FTFSSYAMS

SEQ ID NO: 70 - protein, 15 aa, artificial sequence
  antibody sequence
WVSAIDGSGGSTYYA

SEQ ID NO: 71 - protein, 9 aa, artificial sequence
  antibody sequence
VKGPPVFDY

SEQ ID NO: 72 - protein, 108 aa, artificial sequence
  antibody sequence
SSELTQDPAVSVALGQTVRITCQGDSLRSYYASWYQQKPGQAPVLVIYGKNNRPSGIPDRFSGSSSGNTASLTITGAQAEDEADYYCQSSKWSWDPVVFGGGTKLTVL

SEQ ID NO: 73 - protein, 8 aa, artificial sequence
  antibody sequence
SLRSYYAS

SEQ ID NO: 74 - protein, 11 aa, artificial sequence
  antibody sequence
LVIYGKNNRPS

SEQ ID NO: 75 - protein, 10 aa, artificial sequence
  antibody sequence
QSSKWSWDPV